(19) United States
(12) Patent Application Publication
(10) Pub. No.: US 2016/0003819 A1
(43) Pub. Date: Jan. 7, 2016

(54) RAPID TESTS FOR INSURANCE UNDERWRITING

(71) Applicant: FORCE DIAGNOSTICS, INC, Chicago, IL (US)

(72) Inventors: Michael P. CURRAN, Lake Forest, IL (US); David H. OSTROW, Lake Zurich, IL (US); Brian MULCONREY, Austin, TX (US); Tedd C. DETERMAN, Chicago, IL (US)

(73) Assignee: FORCE DIAGNOSTICS, INC., Chicago, IL (US)

(21) Appl. No.: 14/427,972

(22) PCT Filed: Sep. 11, 2013
(86) PCT No.: PCT/US2013/059282

§ 371 Date: Mar. 12, 2015

Related U.S. Application Data
(63) Continuation-in-part of application No. 13/831,157, filed on Mar. 14, 2013.
(60) Provisional application No. 61/699,891, filed on Sep. 12, 2012.

(51) Int. Cl. G01N 33/558; G01N 33/72; G01N 33/68; G01N 33/569; G01N 33/576

(52) U.S. Cl. CPC G01N 33/558; G01N 33/56988; G01N 33/576; G01N 33/6854; G01N 33/723; G01N 2333/775; G01N 2333/4737; G01N 2333/58

(57) ABSTRACT

The present invention relates to rapid test devices, kits and systems comprising the rapid test devices for assessing at least two analytes in a sample derived from a life or health insurance applicant, said at least two analytes being selected from the group consisting of a human immunodeficiency virus (HIV) antigen (e.g., a HIV polypeptide), a HIV polynucleotide, an anti-HIV antibody, a hepatitis C virus (HCV) antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), pro state-specific antigen (PSA), apolipoprotein A-1 (ApoA1), apolipoprotein B (ApoB), Hemoglobin Ale and high-sensitivity C-reactive protein (hsCRP). The present invention also relates to methods for using the rapid test devices, kits and systems as part of the assessment of the life or health insurance applications.

### I. CROSS-REFERENCE TO RELATED APPLICATIONS

The present application claims priority to U.S. provisional application Ser. No. 61/699,891, filed Sep. 12, 2012, entitled “MULTIPLEX PROGNOSTIC CHIP FOR INSURANCE UNDERWRITING” and U.S. utility application Ser. No. 13/831,157, filed Mar. 14, 2013, entitled “RAPID TESTS FOR INSURANCE UNDERWRITING.” The contents of the above referenced applications are herein incorporated by reference in their entireties.

### II. TECHNICAL FIELD

The present invention relate generally to health screening and insurance underwriting and, more particularly, to kits, devices, systems and methods for assessing the health status of a person.

### III. BACKGROUND OF THE INVENTION

Current insurance underwriting processes require that blood or bodily fluid be collected by a paramedic who either generally comes to a client's residence or requires a client to visit a dedicated facility, for the purpose of drawing multiple vials of blood, via a deep venous puncture, collecting urine sample, Medical Information Bureau (MIB), Attending Physician Statement (APS), completing health questionnaire and measuring blood pressure and body mass index. Once collected, the paramedic sends the body fluid samples to a central clinical or reference laboratory where a battery of prognostic tests are performed, such as standard clinical chemistry and hematology panels on the client's blood. Once all of the tests are conducted, the test information along with the client's Medical Information Bureau (MIB) and Attending Physician Statement (APS) reports and client's application and release of information forms, are collected, collated by and transmitted to the insurance company where a decision is made to insure or not insure the prospective client based on certain results within the panels, blood pressure measurement, body mass index and other reports.

### IV. DISCLOSURE OF THE INVENTION

In one aspect, the present disclosure provides a kit for assessing a life or health insurance applicant, which kit comprises at least two rapid test devices (e.g., singleplex or multiplex rapid test devices), said rapid test devices are configured to assess at least two analytes in a sample derived from a life or health insurance applicant, said at least two analytes being selected from the group consisting of a human immunodeficiency virus (HIV) antigen (e.g., a HIV polypeptide), a HIV polynucleotide, an anti-HIV antibody, a hepatitis C virus (HCV) antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), prostate-specific antigen (PSA), apolipoprotein A-1 (ApoA1), apolipoprotein B (ApoB), Hemoglobin A1c and high-sensitivity C-reactive protein (hsCRP). In some embodiments, the present kits can be used for assessing a life or health insurance applicant at a point of presence.

In another aspect, the present disclosure provides a system for assessing a life or health insurance applicant, which system comprises any of the above kits. In some embodiments, the present systems can be used for assessing a life or health insurance applicant at a point of presence.

In still another aspect, the present disclosure provides a lateral flow test device for assessing a life or health insurance applicant, which device is configured to assess at least two analytes in a sample derived from a life or health insurance applicant, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and said device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to assess said at least two analytes in a sample, and preferably the formation of said detectable signal requires the use of a detectable label. In some embodiments, the present lateral flow test devices can be used for assessing a life or health insurance applicant at a point of presence.

In yet another aspect, the present disclosure provides a system for assessing a life or health insurance applicant, which system comprises any of the above lateral flow test devices. In some embodiments, the present systems can be used for assessing a life or health insurance applicant at a point of presence.

In yet another aspect, the present disclosure provides a method for assessing a life or health insurance applicant, which method comprises: a) assessing the presence, absence and/or amount of at least two analytes in a sample derived from a life or health insurance applicant using a rapid test, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and b) assessing an insurance application from said life or health insurance applicant based on the test results obtained in step a). In some embodiments, the present methods can be used for assessing a life or health insurance applicant at a point of presence.

### V. BRIEF DESCRIPTION OF THE DRAWINGS

The patent or application file contains at least one drawing executed in color. Copies of this patent or patent application publication with color drawings will be provided by the Office upon request and payment of the necessary fee

FIG. 1 illustrates an exemplary lateral flow test device for assessing multiple analytes, e.g., an anti-HIV antibody, an anti-HCV antibody, cotinine and/or nicotine, cocaine and/or benzoylecgonine, and Hemoglobin A1c, using a blood sample.

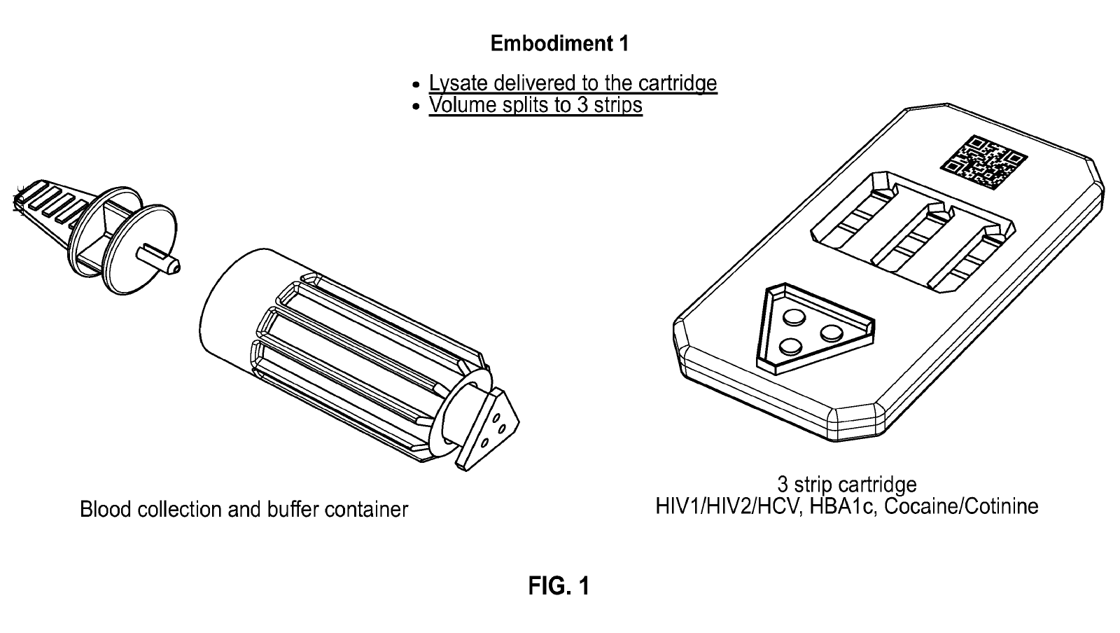

FIG. 2 illustrates exemplary procedures for using the exemplary lateral flow test device illustrated in FIG. 1.

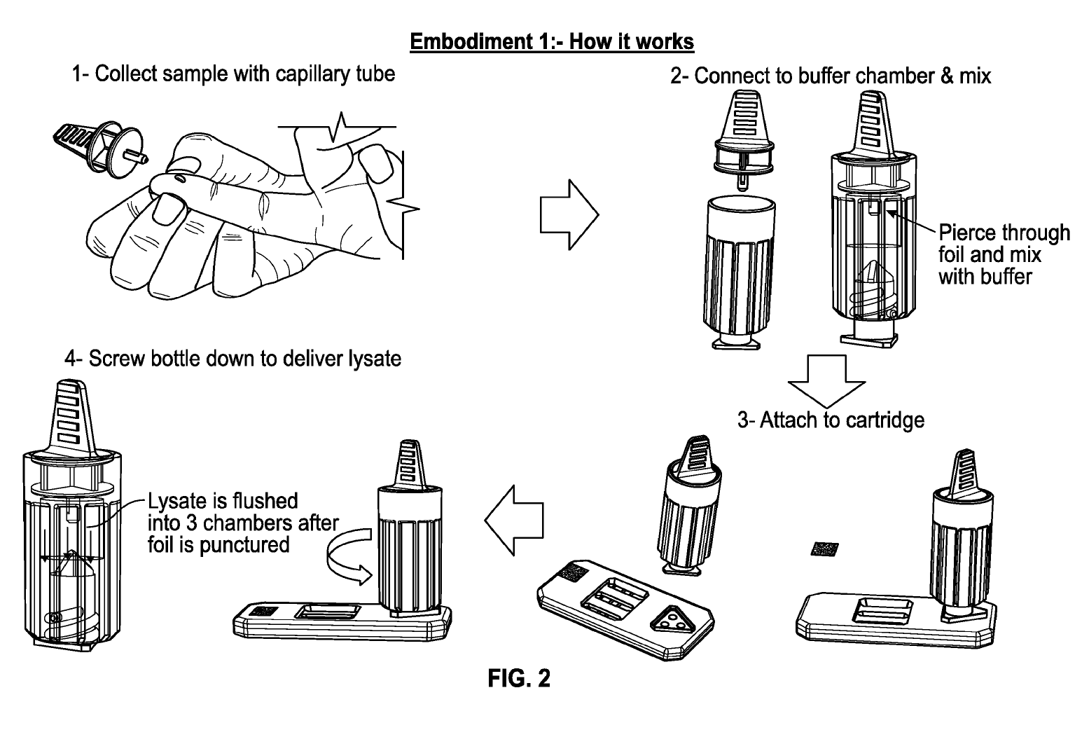

FIG. 3 illustrates an exemplary buffer mixing device and its operation.

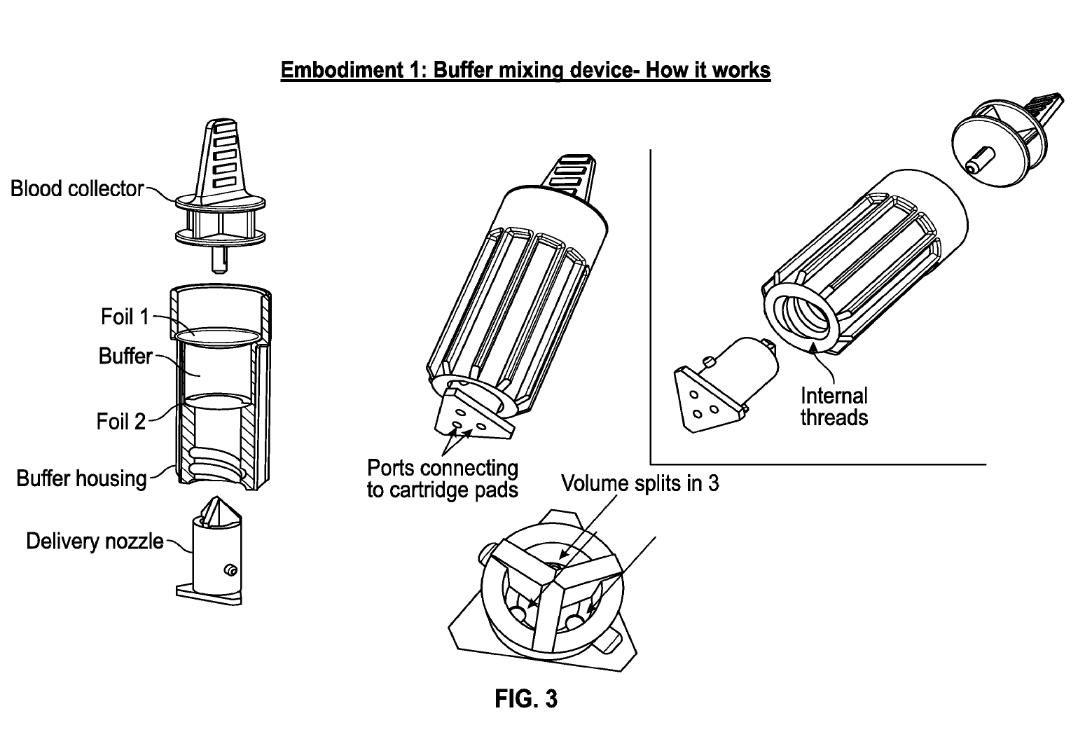

FIG. 4 illustrates an exemplary lysate delivery to test strips.

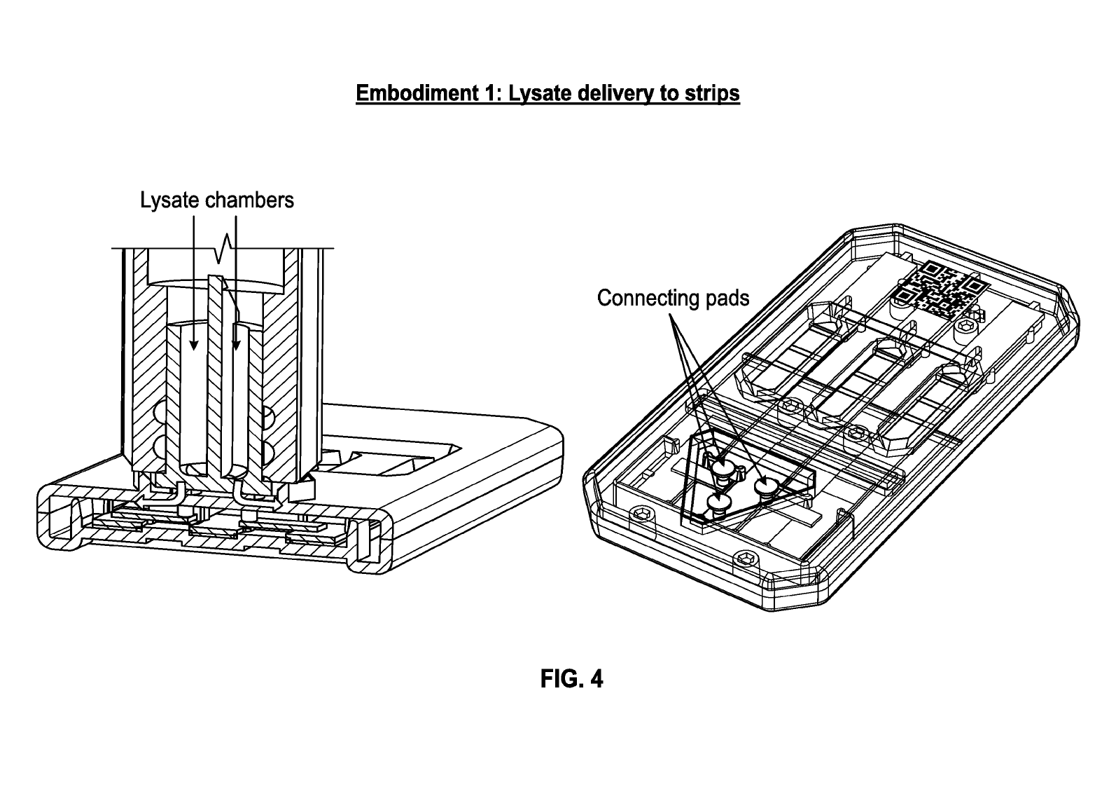

FIG. 5 illustrates another exemplary lateral flow test device for assessing multiple analytes, e.g., an anti-HIV antibody, an anti-HCV antibody, cotinine and/or nicotine, cocaine and/or benzoylecgonine and Hemoglobin A1c, using a whole blood sample.

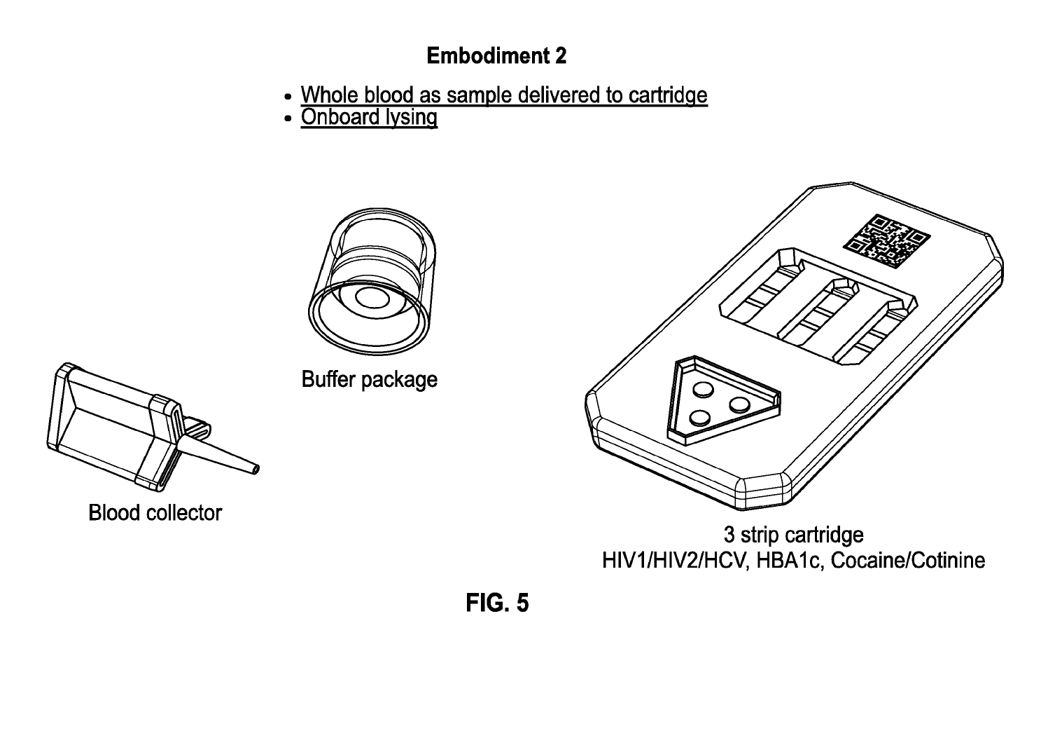

FIG. 6 illustrates exemplary procedures for using the exemplary lateral flow test device illustrated in FIG. 5.

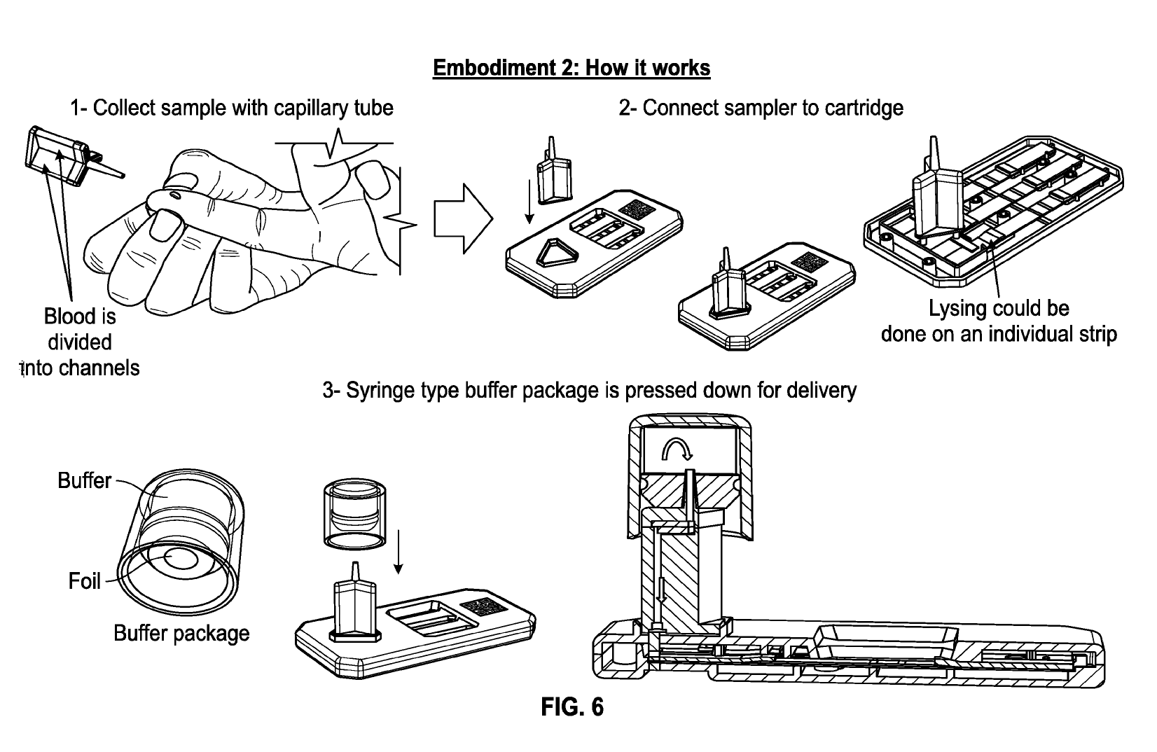

FIG. 7 illustrates an exemplary blood collector and its operation.

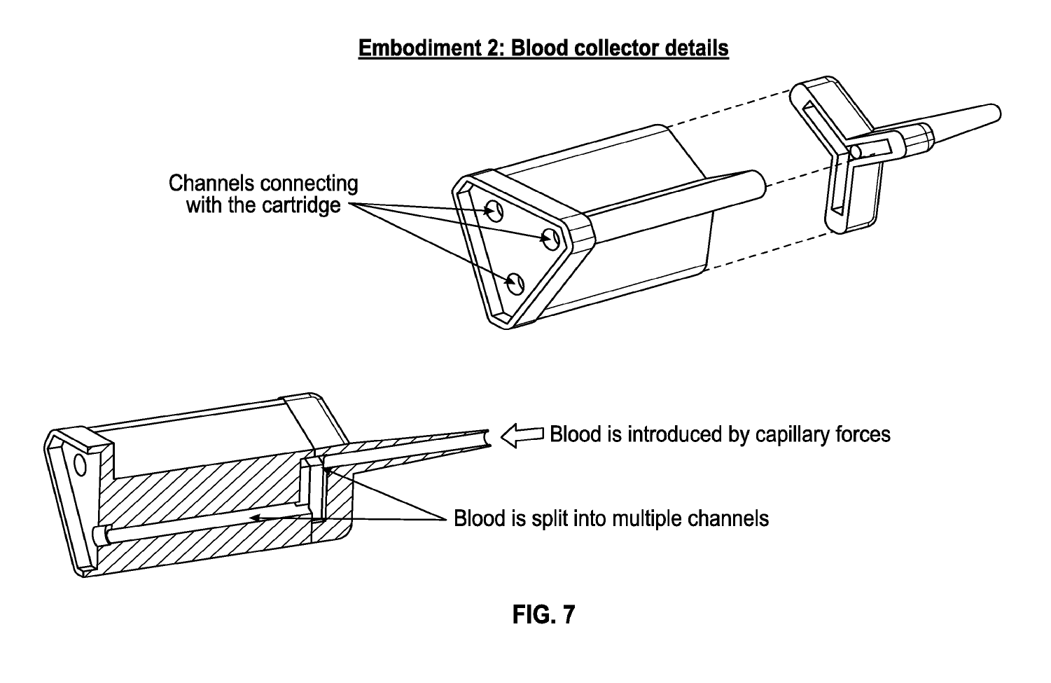

FIG. 8 illustrates an exemplary delivery of a whole blood sample to a cartridge.

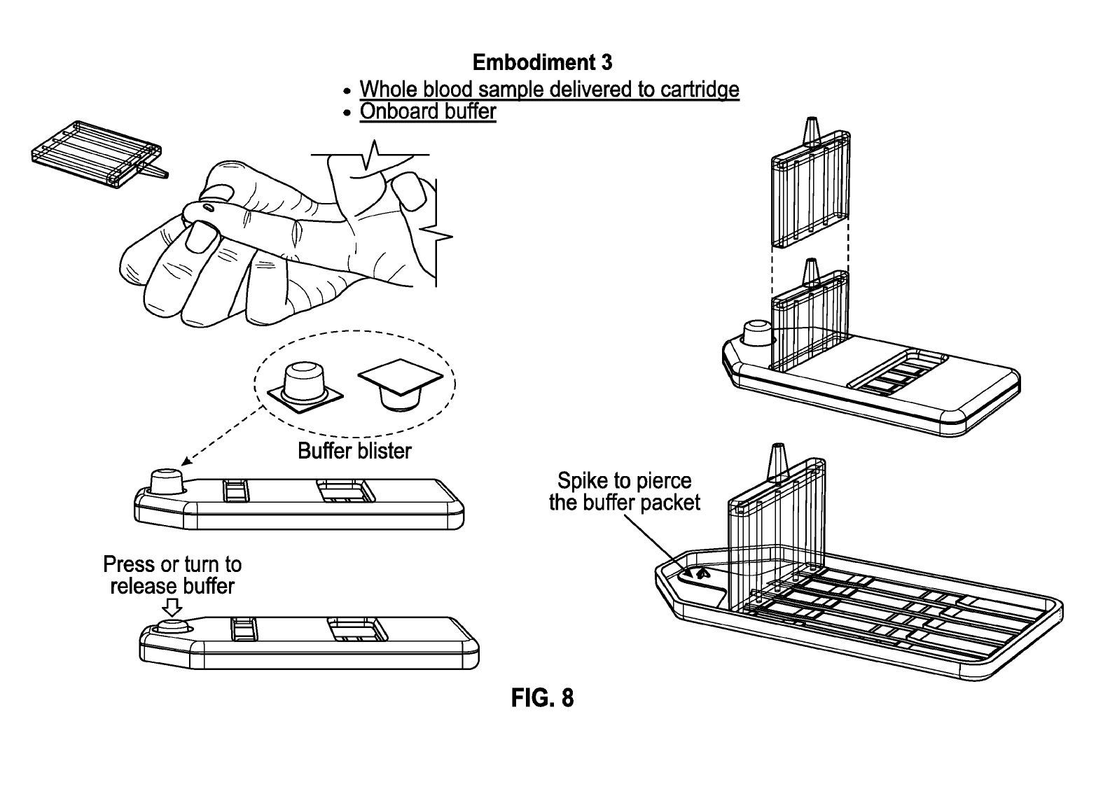

FIG. 9 illustrates still another exemplary lateral flow test device for assessing multiple analytes, e.g., an anti-HIV antibody, an anti-HCV antibody, cotinine and/or nicotine, cocaine and/or benzoylecgonine and Hemoglobin A1c, using a sample volume splitting membrane.

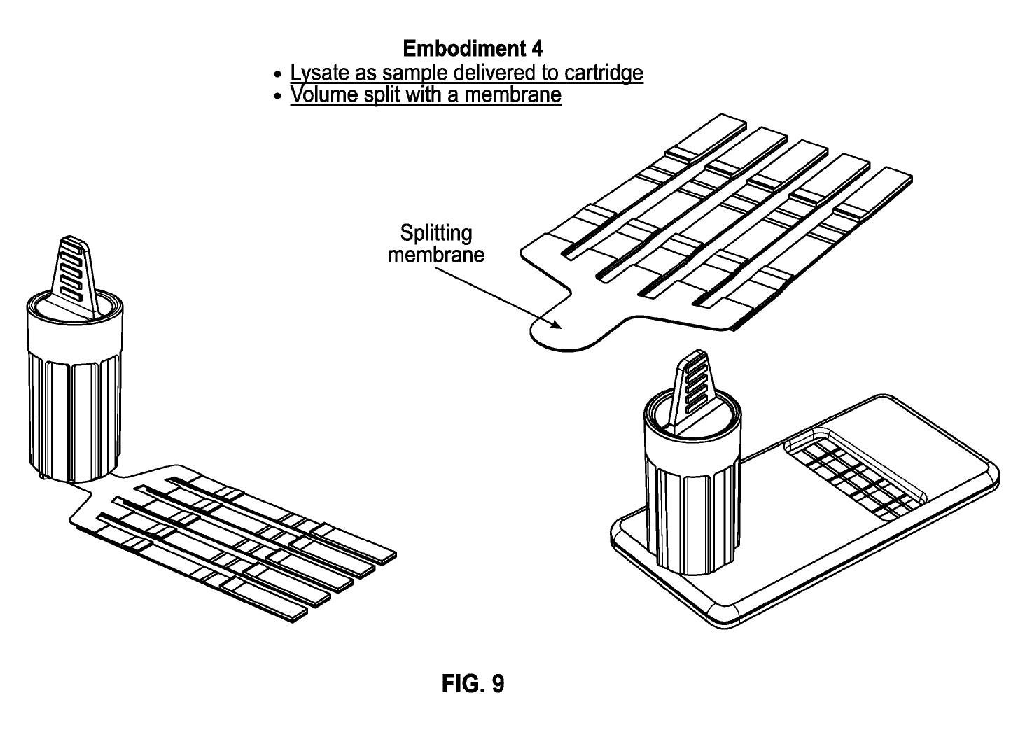

FIG. 10 illustrates an exemplary delivery of a sample to a cartridge containing a sample volume splitting membrane.

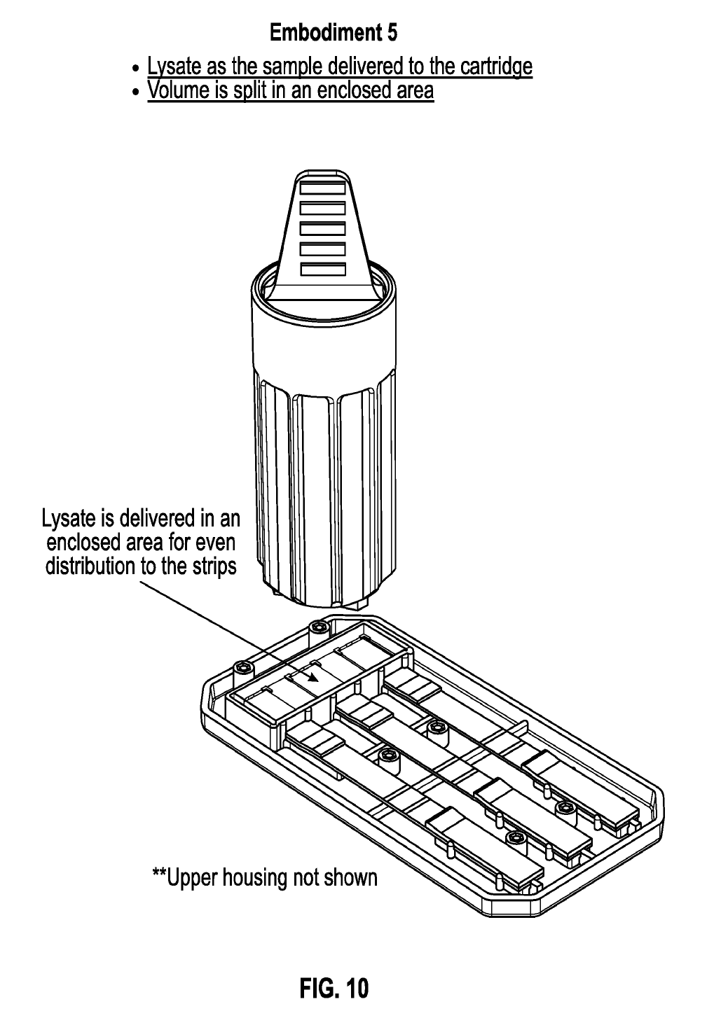

FIG. 11 illustrates an exemplary, current life or health insurance application and underwriting process. As shown in FIG. 11, the current life or health insurance application and underwriting process can take 4-8 weeks to finish.

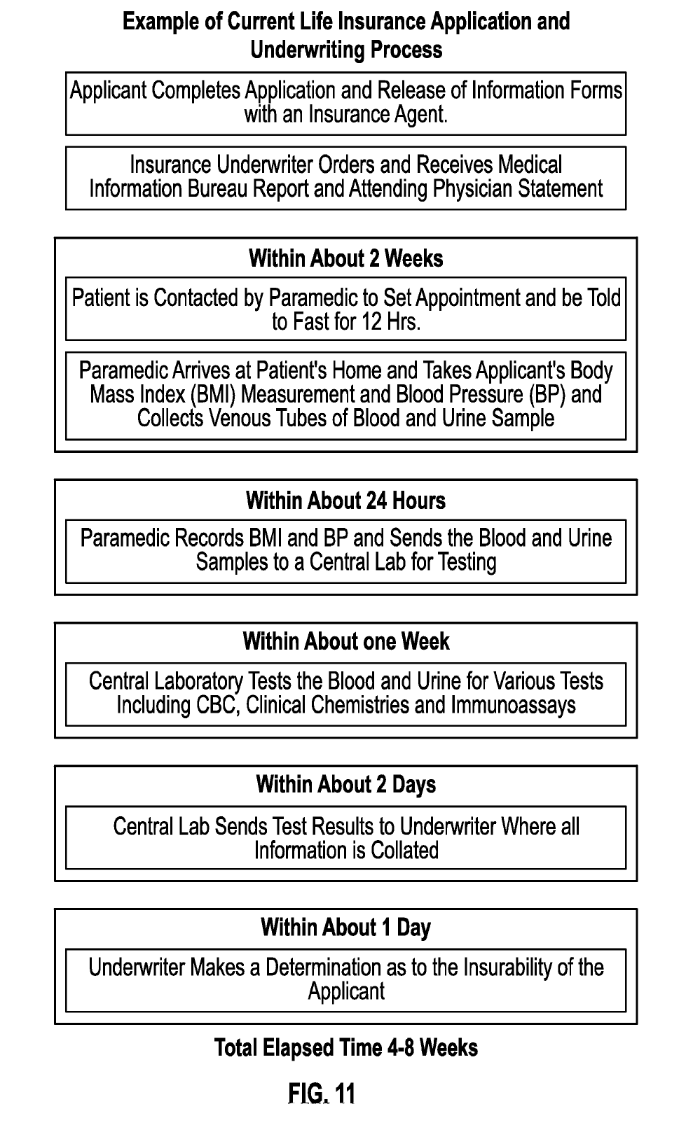

FIG. 12 illustrates an exemplary, fast and inexpensive life or health insurance application process/decision using exemplary kit, device, system and/or method of the present invention. As shown in FIG. 12, the exemplary life or health insurance application and underwriting process can be conducted in about 2 hours.

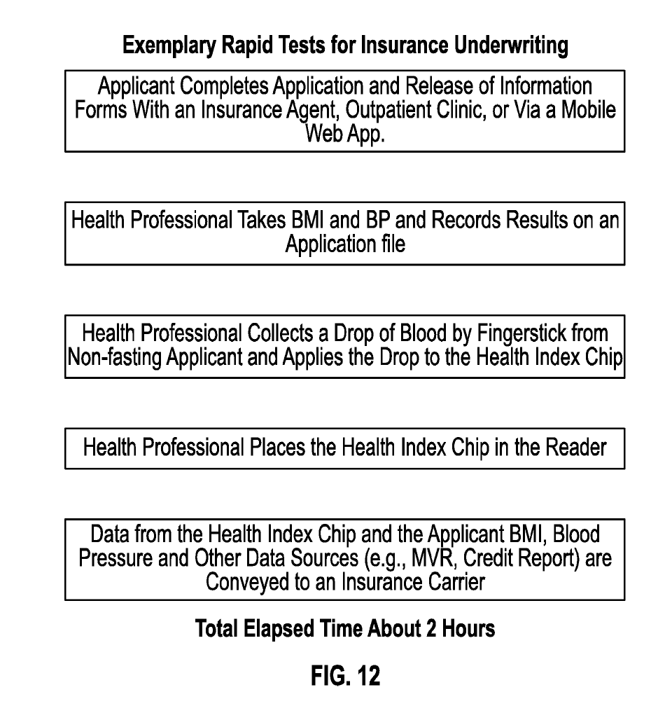

### VI. DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, a “binding reagent” refers to any substance that binds to target or analyte with desired affinity and/or specificity. Non-limiting examples of the binding reagent include cells, cellular organelles, viruses, particles, microparticles, molecules, or an aggregate or complex thereof, or an aggregate or complex of molecules. Exemplary binding reagents can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, an aptamer, a fatty acid, a drug, a drug metabolite and a complex thereof.

As used herein, “antibody” includes not only intact polyclonal or monoclonal antibodies, but also fragments thereof (such as Fab, Fab′, F(ab′)₂, Fv), single chain (ScFv), a diabody, a multi-specific antibody formed from antibody fragments, mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, the term “specifically binds” refers to the specificity of a binding reagent, e.g., an antibody, such that it preferentially binds to a defined analyte or target. Recognition by a binding reagent or an antibody of a particular analyte or target in the presence of other potential targets is one characteristic of such binding. In some embodiments, a binding reagent that specifically binds to an analyte avoids binding to other interfering moiety or moieties in the sample to be tested.

As used herein the term “avoids binding” refers to the specificity of particular binding reagents, e.g., antibodies or antibody fragments. Binding reagents, antibodies or antibody fragments that avoid binding to a particular moiety generally contain a specificity such that a large percentage of the particular moiety would not be bound by such binding reagents, antibodies or antibody fragments. This percentage generally lies within the acceptable cross reactivity percentage with interfering moieties of assays utilizing the binding reagents or antibodies directed to detecting a specific target. Frequently, the binding reagents, antibodies or antibody fragments of the present disclosure avoid binding greater than about 90% of an interfering moiety, although higher percentages are clearly contemplated and preferred. For example, binding reagents, antibodies or antibody fragments of the present disclosure avoid binding about 91%, about 92%, about 93%, about 94%, about 95%, about 96%, about 97%, about 98%, about 99%, and about 99% or more of an interfering moiety. Less occasionally, binding reagents, antibodies or antibody fragments of the present disclosure avoid binding greater than about 70%, or greater than about 75%, or greater than about 80%, or greater than about 85% of an interfering moiety.

As used herein, “mammal” refers to any of the mammalian class of species. Frequently, the term “mammal,” as used herein, refers to humans, human subjects or human patients.

As used herein, the term “subject” is not limited to a specific species or sample type. For example, the term “subject” may refer to a patient, and frequently a human patient. However, this term is not limited to humans and thus encompasses a variety of mammalian species.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “the different liquid flow pathways are substantially parallel to each other” means that the angle between the different liquid flow pathways is at least less than 45 degrees or more than 135 degrees. In some specific embodiments, the angle between the different liquid flow pathways is at about 40, 35, 30, 25, 20, 15, 10, 5, 4, 3, 2, or 1 degree, or the different liquid flow pathways are completely parallel to each other. In other specific embodiments, the angle between the different liquid flow pathways is at about 140, 145, 150, 155, 160, 165, 170, 175, 176, 177, 178, or 179 degrees, or the different liquid flow pathways are completely parallel to each other.

The terms “polypeptide”, “oligopeptide”, “peptide” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucleic Acids Res. 12:203-215.

The terms “homologous”, “substantially homologous”, and “substantial homology” as used herein denote a sequence of amino acids having at least 50%, 60%, 70%, 80% or 90% identity wherein one sequence is compared to a reference sequence of amino acids. The percentage of sequence identity or homology is calculated by comparing one to another when aligned to corresponding portions of the reference sequence.

### B. Kits and Systems for Assessing Life or Health Insurance Applicants

In one aspect, the present disclosure provides for a kit for assessing a life or health insurance applicant, which kit comprises at least two rapid test devices (e.g., singleplex or multiplex rapid test devices), said rapid test devices are configured to assess at least two analytes in a sample derived from a life or health insurance applicant, said at least two analytes being selected from the group consisting of a human immunodeficiency virus (HIV) antigen (e.g., a HIV polypeptide), a HIV polynucleotide, an anti-HIV antibody, a hepatitis C virus (HCV) antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), prostate-specific antigen (PSA), apolipoprotein A-1 (ApoA1), apolipoprotein B (ApoB), Hemoglobin A1c and high-sensitivity C-reactive protein (hsCRP).

Any suitable rapid test devices can be used in the present kits. In some embodiments, the present kits comprise singleplex rapid test devices. In other embodiments, the present kits comprise multiplex rapid test devices. In still other embodiments, the present kits comprise both singleplex and multiplex rapid test devices.

The present kits can be used for assessing a life or health insurance applicant at any suitable location. In some embodiments, the present kits can be used for assessing a life or health insurance applicant at a point of presence, e.g., at the location where a sample from an applicant is obtained, an insurance application is submitted and/or accepted, and/or an insurance decision is made. Exemplary point of presence can be a residential place, an office, e.g., a medical office or an insurance office, a retail location or a store, e.g., a pharmacy store or a supermarket, a clinical laboratory, or a health station or kiosk. Point of presence does not mean that the rapid tests must be conducted in the presence of a life or health insurance applicant. In some embodiments, the rapid tests can be conducted in the presence of a life or health insurance applicant. In other embodiments, the rapid tests can be conducted in the absence of a life or health insurance applicant. For example, after a sample is obtained from a life or health insurance applicant, the life or health insurance applicant may leave the location where the sample from the applicant is obtained before or during the rapid tests are conducted, and the applicant can be notified of the insurance decision subsequently.

The present kit can comprise any suitable number of the rapid test devices. For example, the present kit can comprise 2, 3, 4, 5, 6, 7, 8, 9, 10, 11, 12, 13 or 14 rapid test devices. In some embodiments, the present kit comprises at least 3, 4, or 5 rapid test devices, and wherein the rapid test devices are configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP. In other embodiments, the present kit comprises at least 5 rapid test devices, and wherein the rapid test devices are configured to assess 5 analytes selected from the group consisting of an anti-HIV antibody, an anti-HCV antibody, cotinine and/or nicotine, cocaine and/or benzoylecgonine, and Hemoglobin A1c.

The present kit can comprise any suitable type(s) of the rapid test devices. In some embodiments, at least one of the rapid test devices in the present kit can be a lateral flow test device, a flow through test device, a microfluidic test device, an immunochromatography test device, a single use cartridge or test device, a disposable test device, a self-contained test device, a point of care test device, a microsphere based test device (See e.g., U.S. Pat. No. 7,718,262), a nanoparticle based test device (See e.g., U.S. Pat. No. 7,773,790), a test strip device (See e.g., U.S. Pat. No. 6,773,671), a spot test device (See e.g., U.S. Pat. No. 6,498,010), a centrifugal device (See e.g., U.S. Pat. No. 8,338,191), a pump based test device, or a flow control device for assays (See e.g., WO 2011/124991 A2 and U.S. provisional application Ser. No. 61/321,707). In other embodiments, at least 2, 3, 4, 5, 6, 7, 8, 9, 10, 11, 12, 13 or 14 of the rapid test devices in the present kit can be a lateral flow test device, a flow through test device, a microfluidic test device, an immunochromatography test device, a single use cartridge or test device, a disposable test device, a self-contained test device, a point of care test device, a microsphere based test device, a nanoparticle based test device, a test strip device, a spot test device, a centrifugal device, or a pump based test device.

The rapid test devices in the present kit can be used to conduct a test within any suitable time period, e.g., in about 12, 11, 10, 9, 8, 7, 6, 5, 4, 3, 2 or 1 hour, 50 minutes, 40 minutes, 30 minutes, 20 minutes, or 10 minutes, from the time a sample is obtained from an applicant. In some embodiments, at least one of the rapid test devices can be used to conduct a test within any suitable time period, e.g., in about 12, 11, 10, 9, 8, 7, 6, 5, 4, 3, 2 or 1 hour, 50 minutes, 40 minutes, 30 minutes, 20 minutes, or 10 minutes, from the time a sample is obtained from an applicant. In other embodiments, at least 2, 3, 4, 5, 6, 7, 8, 9, 10, 11, 12, 13 or 14 of the rapid test devices can be used to conduct a test within any suitable time period, e.g., in about 12, 11, 10, 9, 8, 7, 6, 5, 4, 3, 2 or 1 hour, 50 minutes, 40 minutes, 30 minutes, 20 minutes, or 10 minutes, from the time a sample is obtained from an applicant.

In some embodiments, the present kit can comprise at least 2, 3, 4, 5, 6, 7, 8, 9, 10, 11, 12, 13 or 14 lateral flow test devices. In other embodiments, the present kit can comprise at least 3, 4, or 5 lateral flow test devices, and wherein the lateral flow test devices are configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP. In still other embodiments, the present kit can comprise at least 5 lateral flow test devices, and wherein the lateral flow test devices are configured to assess 5 analytes selected from the group consisting of an anti-HIV antibody, an anti-HCV antibody, cotinine and/or nicotine, cocaine and/or benzoylecgonine, and Hemoglobin A1c.

The readout signal from tests using the present kits can be assessed by any suitable means. In some embodiments, the readout signal from tests using the present kits can be assessed by using any suitable label-free detection methods. Exemplary label-free detection methods include the detection methods based on surface plasmon resonance, bio-layer interferometry, epic technology, quartz crystal microbalance, electrical impedance and microcalorimetry. The label-free detection methods disclosed and/or claimed in the following U.S. Pat. Nos. can also be used: U.S. Pat. Nos. 7,531,786, 7,737,392, 7,742,662, 7,790,406, 7,863,052, 7,955,883, 7,960,170, 7,968,836 and 8,145,434. In some embodiments, the readout signal from tests using the present kits can be assessed by using any suitable detection methods using a detectable label.

In some embodiments, at least 1, 2, 3, 4, 5, 6, 7, 8, 9, 10, 11, 12, 13 or 14 lateral flow test devices can comprise a porous matrix that comprises a test zone on the porous matrix, the test zone comprising a test reagent that binds to an analyte or another binding reagent that binds to the analyte, or is an analyte or an analyte analog that competes with an analyte in a sample for binding to a binding reagent for the analyte, a liquid sample flows laterally along the test device and passes the test zone to form a detectable signal to indicate presence, absence and/or amount of the analyte in the liquid sample, and the formation of the detectable signal requires the use of a detectable label.

The rapid test devices, e.g., lateral flow test devices, can use any suitable assay format, e.g., a sandwich assay or a competitive assay format. In some embodiments, the rapid test devices, e.g., lateral flow test devices, can be used in a sandwich assay for the analyte and wherein the test reagent at the test zone binds to the analyte, and a second binding reagent that comprises a detectable label and binds to the analyte and is used. In other embodiments, the rapid test devices, e.g., lateral flow test devices, can be used in a sandwich assay for the analyte and wherein the test reagent at the test zone binds to the analyte, and a second binding reagent that comprises a detectable label and binds to another binding reagent that binds to an analyte is used.

In some embodiments, the rapid test devices, e.g., lateral flow test devices, can be used in a competitive assay for the analyte and wherein the test reagent at the test zone is an analyte or an analyte analog, a second binding reagent that comprises a detectable label and binds to the analyte is used, and the analyte or an analyte analog at the test zone competes with an analyte in the sample for binding to the second binding reagent. In other embodiments, the rapid test devices, e.g., lateral flow test devices, can be used in a competitive assay for the analyte and wherein the test reagent at the test zone is an analyte or an analyte analog, a second binding reagent that comprises a detectable label and binds to another binding reagent that binds to an analyte is used, and the analyte or an analyte analog at the test zone competes with an analyte in the sample for binding to the binding reagent that is bound to the second binding reagent. In still other embodiments, the rapid test devices, e.g., lateral flow test devices, can be used in a competitive assay for the analyte and wherein the test reagent at the test zone is a binding reagent that binds to the analyte, an analyte or analyte analog linked to a detectable label is used, and the analyte or an analyte analog linked to a detectable label competes with an analyte in the sample for binding to the binding reagent at the test zone.

The present kits can be used to assess any suitable types of analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP. For example, the analyte can be a polypeptide, a polynucleotide or a small molecule. In some embodiments, the analyte is a polypeptide or a small molecule, and the test reagent and/or binding reagent that binds to the analyte is a binding reagent for the analyte. Preferably, the binding reagent binds specifically to the analyte. Any suitable binding reagents can be used. In some embodiments, the binding reagent can be an antibody that binds to the polypeptide or small molecule. Preferably, the antibody binds specifically to the analyte. In some embodiments, the analyte is a polynucleotide, and the binding reagent is another polynucleotide that is complementary or substantially complementary with the analyte polynucleotide. Any suitable HIV antigen (e.g., a HIV polypeptide) or HIV polynucleotide can be detected. For example, a HIV antigen (e.g., a HIV polypeptide) or HIV polynucleotide from HIV-1, HIV-2 or both can be detected. Any suitable antibody to a HIV antigen can be detected. For example, an antibody to a HIV-1 antigen, a HIV-2 antigen or both can be detected. In some embodiments, an antibody to Group O antigen from HIV-1, HIV-2 or both can be detected. The rapid tests can be based on any suitable test principle such as affinity chromatography. For example, Hemoglobin A1c can be detected using boronate affinity or immuno affinity methods.

The rapid test devices, e.g., lateral flow test devices, can comprise any suitable matrix. In some embodiments, the matrix comprises nitrocellulose, glass fiber, polypropylene, polyethylene (preferably of very high molecular weight), polyvinylidene fluoride, ethylene vinyl acetate, acrylonitrile and/or polytetrafluoro-ethylene. The matrix can be in any suitable shape. In some embodiments, the matrix is in the form a strip or a circle. The matrix can comprise any suitable number of elements. In some embodiments, the matrix is a single element or comprises multiple elements.

The rapid test devices, e.g., lateral flow test devices, can further comprise a sample application element upstream from and in fluid communication with the matrix. The rapid test devices, e.g., lateral flow test devices, can further comprise a liquid absorption element downstream from and in fluid communication with the matrix. The rapid test devices, e.g., lateral flow test devices, can further comprise a control zone comprising means for indicating proper flow of the liquid sample and/or a valid test result.

In some embodiments, at least a portion of the matrix is supported by a solid backing.

In some embodiments, a substance is dried on a portion of the matrix upstream from the test zone, the dried substance being capable of being moved by a liquid sample and/or a further liquid to the test zone and/or a control zone to generate a detectable signal, the dried substance being at least one of the second binding reagent that binds to the analyte, the second binding reagent that binds to another binding reagent that binds to the analyte, the analyte or the analyte analog, each of the second binding reagent, analyte or analyte analog comprises a detectable label.

The substance can be located at any suitable location. In some embodiments, the substance is dried on a conjugate element that is upstream from the test zone. In other embodiments, the substance is located downstream from a sample application place on the test device. In still other embodiments, the substance is located upstream from a sample application place on the test device.

Any suitable detectable label can be used. In some embodiments, the detectable label is a soluble label, e.g., a soluble enzyme or fluorescent label. In other embodiments, the detectable label is a particle label. The particle label can be a visible or a non-visible particle label. Any visible particle label can be used. In some embodiments, the visible particle label can be a colloidal gold label, a latex particle label, a nanoparticle label and a quantum dot label. Any non-visible particle label can be used. In some embodiments, the non-visible particle label can be a fluorescent particle.

In some embodiments, the substance can be dried in the presence of a material that: a) stabilizes the dried substance; b) facilitates solubilization or re-suspension of the dried substance in a liquid; and/or c) facilitates mobility of the dried substance. Any suitable material can be used. For example, a material can be a protein, a peptide, a polynucleotide a polysaccharide, a sugar, a polymer, a gelatin and/or a detergent.

An analyte and/or the substance can be transported to the test zone using any suitable liquid. In some embodiments, a sample liquid alone is used to transport the analyte and/or the substance to the test zone. In other embodiments, a developing liquid is used to transport the analyte and/or the substance to the test zone. In still other embodiments, a sample treatment liquid can be employed to lyse, solubilize and/or transport a sample analyte.

The rapid test devices, e.g., lateral flow test devices, can further comprise a housing that covers at least a portion of the test device, wherein the housing comprises a sample application port to allow sample application upstream from or to the test zone and an optic opening around the test zone to allow signal detection at the test zone. In some embodiments, the housing covers the entire test device. In other embodiments, at least a portion of the sample receiving portion of the matrix or the sample application element is not covered by the housing and a sample is applied to the portion of the sample receiving portion of the matrix or the sample application element outside the housing and then transported to the test zone.

The rapid test devices, e.g., lateral flow test devices, can be configured to receive any suitable type of sample. In some embodiments, at least one of the rapid test devices or lateral flow test devices is configured to receive one type of sample, and another rapid test device or lateral flow test device is configured to receive a different type of sample. In other embodiments, at least one of the rapid test devices or lateral flow test devices is configured to receive a blood sample and another rapid test device or lateral flow test device is configured to receive a saliva sample.

The rapid test devices, e.g., lateral flow test devices, can be used to assess an analyte qualitatively, quantitatively or semi-quantitatively. In some embodiments, at least one of the lateral flow test devices is configured to assess an analyte qualitatively. In other embodiments, at least one of the lateral flow test devices is configured to assess an analyte quantitatively or semi-quantitatively.

The rapid test devices, e.g., lateral flow test devices, can be used to assess any suitable number of analyte(s). In some embodiments, at least one of the lateral flow test devices is configured to assess at least two analytes.

The present kits can further comprise a solid medium for collecting, drying and/or storing a sample derived from a life or health insurance applicant wherein said collected, dried and/or stored sample can be used in a test.

Any suitable solid medium can be used in the present kits. In some embodiments, the solid medium is a porous solid medium. Any suitable porous solid medium can be used. For example, the porous solid medium can comprise filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene. In other embodiments, the solid medium is a non-porous solid medium. Any suitable non-porous solid medium can be used. For example, the non-porous solid medium can comprise a plastic material or glass slide.

In some embodiments, the solid medium can be contained in a container or device can be at least a part of a surface of a container. Any suitable container can be used. For example, the container can be a tube or a microtiter plate.

Any suitable sample derived from a life or health insurance applicant can be collected, dried and/or stored on or in the solid medium of the present kits. In some embodiments, the dried sample is a dried body fluid sample, e.g., a whole blood, a serum, a plasma, a fresh blood, a blood not containing an anti-coagulant, a urine and a saliva sample. In some embodiments, the dried sample comprises an analyte, e.g., a polypeptide, a polynucleotide or a small molecule.

In some embodiments, the sample can be collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content. Any such suitable material can be used. For example, the material can be a protein, a peptide, a polynucleotide, a polysaccharide, a sugar, a polymer, a gelatin and a detergent.

In some embodiments, the present disclosure provides for kits wherein the solid medium comprises a collected, dried and/or stored sample.

The sample can be collected, dried and/or stored on or in a solid medium in any suitable manner or form. For example, the sample can be dried on a solid medium as a dried spot.

The sample can be stored and remain useable and/or stable for any suitable period of time. For example, the sample can be stored for a period of time during which the insurance application and/or decision can be reviewed, reassessed and/or contested. In some embodiments, the dried sample can be stored for a short term, e.g., about 1, 2, 3, 4, 5, 6, 7, 8, 9 or 10 days. In other embodiments, the dried sample can be stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

The collected, dried and/or stored sample can be used for any suitable purpose. In some embodiments, the collected, dried and/or stored sample can be retested for an analyte that has been tested before for assessing a life or health insurance applicant from which the sample is derived. In other embodiments, the collected, dried and/or stored sample can be tested for an analyte that has not been tested before. In still other embodiments, the collected, dried and/or stored sample can be tested for confirmation of the presence or absence or concentration of an analyte. In yet other embodiments, the collected, dried and/or stored sample can be tested for assessing an identifier, e.g., a personal identifier, of the life or health insurance applicant.

The present kits can further comprise a means for collecting additional sample from a life or health insurance applicant to be stored on a solid medium. Any suitable means can be used. For example, the means can comprise a device for collecting a sample to be dried or stabilized for storage purpose. The dried or stabilized sample can be used for any suitable purposes. For example, the dried or stabilized sample can be suitable for an optional test that can be completed at a later time, e.g., an optional test for assessing optional insurance coverage.

In some embodiments, in the event of a positive result with the rapid insurance tests that most likely may be a public health issue or disputed issue by the applicant, a sample at the time of collection can be saved through a dried body fluid spot that can be used to re-test the applicant or patient or without calling he/she back to the retail pharmacy or urgent care clinic to re-sample. The dried spot sample can be re-tested immediately, sent for further analysis such as confirmation or saved for applicant identification.

In some embodiments, a sample at the time of collection can be saved through a dried body fluid spot using any suitable devices and methods know in the art. Such exemplary devices and methods include methods of drying blood for stable glucose testing disclosed in U.S. Pat. No. 5,204,267; an apparatus for preparing and processing dried blood spots for HIV testing on a test card and punching out an exact area that represents a quantifiable amount of blood residue disclosed in U.S. Pat. Nos. 5,641,682, 5,862,729 and 6,171,868; devices and methods for preserving and extracting nucleic acids from cells from whole blood dried blood spots for nucleic acid testing disclosed in U.S. Pat. No. 7,927,798; devices and methods for quantitative recovery of physiologically important enzymes from body fluids including dried blood and saliva spots disclosed in U.S. Pat. No. 6,756,230; and an apparatus for collecting and drying body fluid samples for later analysis disclosed in U.S. Pat. No. 7,638,099.

In some embodiments, the present disclosure provides for a kit for rapid insurance testing containing a filter paper or apparatus for drying a blood sample forming a dried blood spot or other body fluid dried spot for longer term storage of sample for retest or confirmatory or identification testing. In other embodiments, the present disclosure provides for a kit containing a body fluid collection apparatus or vessel for collecting extra sample for short term retesting or confirmatory testing. In still other embodiments, the present disclosure provides for a method for rapid insurance testing that includes taking an extra fingerstick blood sample or diluted blood sample, or extra other body fluid or diluted body fluid and drying on filter paper or apparatus to dry the sample for later testing without recalling the patient or applicant. The dried body fluid spot can be used for any suitable purpose, e.g., to identify the applicant using biomolecular means to prove originality of the sample.

In another aspect, the present disclosure provides for a system for assessing a life or health insurance applicant, which system comprises any of the above kits.

The present system can further comprise an instruction for using the system for assessing mortality and/or morbidity risk of the life or health insurance applicant, and/or making a decision on the insurance or health application from the life or health insurance applicant.

The present system can further comprise a means for assessing an additional health indicator of the life or health insurance applicant. Any suitable means can be used. In some embodiments, the means is used to assess a blood pressure, a body mass index and/or alcohol consumption or alcoholism of the life or health insurance applicant. Any suitable means for detecting alcohol consumption or alcoholism can be used. For example, a number of questionnaires that are used to detect or diagnosis alcoholism can be included in the present system. In another example, test devices and/or reagents for detecting alcohol consumption or alcoholism, such as test devices and/or reagents for detecting gammaglutamyl transferase (ggt) and other liver function tests, mean corpuscular volume (MCV), folate/b12, magnesium, or carbohydrate deficient form of transferrin (CDT), can be included in the present system.

The present system can further comprise a means for assessing an identifier of the life or health insurance applicant. Any suitable means can be used. In some embodiments, the means is an iris scan, a fingerprinting device, a haplotyping device, or a nucleic acid, e.g., DNA, sequencer, or a means for assessing voice identification, photograph, electronic signature, or photo identification. In some embodiments, the nucleic acid, e.g., DNA, sequence information from an applicant can be used to verify the identity of the applicant. In some embodiments, the nucleic acid, e.g., DNA, sequence information from an applicant is not used or considered in the insurance underwriting.

In some embodiments, the present system can further comprise a means for obtaining at least two different types of samples from the life or health insurance applicant. In other embodiments, the present system can further comprise a means for collecting more than one finger stick of blood from the life or health insurance applicant to increase the volume of blood sample to be able to run multiple assays on the blood sample. In still other embodiments, the present system can further comprise a finger stick device that simultaneously makes at least two punctures at once to increase the amount of blood sample volume needed to perform multiple assays on the blood sample.

The present system can further comprise a machine-readable information, e.g., a bar code. The machine-readable information can be located at any suitable location, e.g., as a part of the rapid test device, or as a separate component in the kit. The machine-readable information can be stored in any suitable storage medium, e.g., a RFID device.

The present system can further comprise a reader to assess the detectable signal. Any suitable reader can be used. For example, the reader can be a fluorescent reader, a colorimetric reader, or a luminescent reader. Any suitable fluorescent reader can be used. For example, the fluorescent reader can be a laser based or a light emitting diode (LED) based fluorescent reader. The present system can further comprise a means for recording, storing and/or sending test results in an electronic format, and/or a software program and/or algorithm that collates and/or optimizes all data inputs such as assay results, BMI, application, release form, attending physician statement (APS), medical information bureau (MIB) record, motor vehicle record (MVR), credit report and prescription or transaction history record into one electronically transferrable or printable file, and/or an apparatus, software and/or algorism for forensic voice analysis that detects emotions in the human voice. Any suitable apparatus, software or algorism for forensic voice analysis that detects emotions in the human voice can be used. See e.g., U.S. Pat. No. 7,165,033.

The present systems can further comprise a solid medium for collecting, drying and/or storing a sample derived from a life or health insurance applicant wherein said collected, dried and/or stored sample can be used in a test.

Any suitable solid medium can be used in the present kits. In some embodiments, the solid medium is a porous solid medium. Any suitable porous solid medium can be used. For example, the porous solid medium can comprise filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene. In other embodiments, the solid medium is a non-porous solid medium. Any suitable non-porous solid medium can be used. For example, the non-porous solid medium can comprise a plastic material or glass slide.

In some embodiments, the solid medium can be contained in a container or device can be at least a part of a surface of a container. Any suitable container can be used. For example, the container can be a tube or a microtiter plate.

Any suitable sample derived from a life or health insurance applicant can be collected, dried and/or stored on or in the solid medium of the present systems. In some embodiments, the dried sample is a dried body fluid sample, e.g., a whole blood, a serum, a plasma, a fresh blood, a blood not containing an anti-coagulant, a urine and a saliva sample. In some embodiments, the dried sample comprises an analyte, e.g., a polypeptide, a polynucleotide or a small molecule.

In some embodiments, the sample can be collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content. Any such suitable material can be used. For example, the material can be a protein, a peptide, a polynucleotide, a polysaccharide, a sugar, a polymer, a gelatin and a detergent.

In some embodiments, the present disclosure provides for systems wherein the solid medium comprises a collected, dried and/or stored sample.

The sample can be collected, dried and/or stored on or in a solid medium in any suitable manner or form. For example, the sample can be dried on a solid medium as a dried spot.

The sample can be stored and remain useable and/or stable for any suitable period of time. For example, the sample can be stored for a period of time during which the insurance application and/or decision can be reviewed, reassessed and/or contested. In some embodiments, the dried sample can be stored for a short term, e.g., about 1, 2, 3, 4, 5, 6, 7, 8, 9 or 10 days. In other embodiments, the dried sample can be stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

The collected, dried and/or stored sample can be used for any suitable purpose. In some embodiments, the collected, dried and/or stored sample can be retested for an analyte that has been tested before for assessing a life or health insurance applicant from which the sample is derived. In other embodiments, the collected, dried and/or stored sample can be tested for an analyte that has not been tested before. In still other embodiments, the collected, dried and/or stored sample can be tested for confirmation of the presence or absence or concentration of an analyte. In yet other embodiments, the collected, dried and/or stored sample can be tested for assessing an identifier, e.g., a personal identifier, of the life or health insurance applicant.

In some embodiments, in the event of a positive result with the rapid insurance tests that most likely may be a public health issue or disputed issue by the applicant, a sample at the time of collection can be saved through a dried body fluid spot that can be used to re-test the applicant or patient or without calling he/she back to the retail pharmacy or urgent care clinic to re-sample. The dried spot sample can be re-tested immediately, sent for further analysis such as confirmation or saved for applicant identification.

In some embodiments, a sample at the time of collection can be saved through a dried body fluid spot using any suitable devices and methods know in the art. Such exemplary devices and methods include methods of drying blood for stable glucose testing disclosed in U.S. Pat. No. 5,204,267; an apparatus for preparing and processing dried blood spots for HIV testing on a test card and punching out an exact area that represents a quantifiable amount of blood residue disclosed in U.S. Pat. Nos. 5,641,682, 5,862,729 and 6,171,868; devices and methods for preserving and extracting nucleic acids from cells from whole blood dried blood spots for nucleic acid testing disclosed in U.S. Pat. No. 7,927,798; devices and methods for quantitative recovery of physiologically important enzymes from body fluids including dried blood and saliva spots disclosed in U.S. Pat. No. 6,756,230; and an apparatus for collecting and drying body fluid samples for later analysis disclosed in U.S. Pat. No. 7,638,099.

The present systems can further comprise a means for collecting additional sample from a life or health insurance applicant to be stored on a solid medium. Any suitable means can be used. For example, the means can comprise a device for collecting a sample to be dried or stabilized for storage purpose. The dried or stabilized sample can be used for any suitable purposes. For example, the dried or stabilized sample can be suitable for an optional test that can be completed at a later time, e.g., an optional test for assessing optional insurance coverage.

The system can further comprise a device or instrument for conducting a thyroid test, a thyroid panel test, a test for liver marker, a metabolic panel test, a lipid test, a test for blood count, an autoimmune test, and/or an infectious disease test on a sample from a life or health insurance applicant.

The present systems can comprise any suitable thyroid test. For example, the present systems can comprise a thyroid test that assesses thyroid function using thyroid-stimulating hormone (TSH). The present systems can comprise any suitable thyroid panel test. For example, the present systems can comprise a thyroid panel test that assesses TSH, thyroxine (T4), and Triiodothyronine (T3).

The present systems can comprise any suitable test for liver function. For example, the present systems can comprise a test for liver disease by measuring Gamma-glutamyltransferase (GGT), antinuclear antibodies (ANA), alkaline phosphatase (ALP), albumin, and/or prothrombin.

The present systems can comprise any suitable metabolic panel test. For example, the present systems can comprise a metabolic panel test that assesses acid/base balance, glucose, sodium, potassium, blood urea nitrogen (BUN), creatinine, and/or blood gases.

The present systems can comprise any suitable lipid test. For example, the present systems can comprise a lipid test that assesses apolipoprotein A-I (ApoA1) and/or apolipoprotein B (ApoB).

The present systems can comprise any suitable test for blood diseases. For example, the present systems can comprise a complete blood count that comprises hematocrit, hemoglobin, mean corpuscular volume (MCV), lymphocyte count, white blood cell (WBC) count, platelets, and reticulocytes.

The present systems can comprise any suitable autoimmune test. For example, the present systems can comprise an autoimmune test that assesses ANA and/or rheumatoid factor (RF).

The present systems can comprise any suitable infectious disease test. For example, the present systems can comprise an infectious disease test that assesses a maker for tuberculosis (TB), malaria, and/or dengue.

The present systems can comprise any suitable device or instrument for conducting a thyroid test, a thyroid panel test, a test for a liver marker, a metabolic panel test, a lipid test, a test for blood count, an autoimmune test, and/or an infectious disease test on a sample from a life or health insurance applicant. For example, the device or instrument can be a clinical chemistry analyzer, a hematology analyzer, or a hand-held device or instrument. The device or instrument can conduct a test within any suitable period of time. For example, the device or instrument can conduct a test within about 1 hour, 2 hours, 4 hours, 8 hours, 16 hours, or 24 hours.

In some embodiments, further testing can be undertaken at the retail clinic, retail pharmacy such as Walgreens, or urgent care clinic, and can be accomplished in under one-hour time. Such exemplary testing can include a thyroid test (TSH) or thyroid panels (TSH, T4, T3), additional liver testing (GGT, ANA, ALP, albumin, prothrombin), metabolic panels (acid/base balance, glucose, sodium, potassium, BUN, creatinine, blood gases), additional lipid tests (ApoA1, ApoB), and complete blood count (hematocrit, hemoglobin, MCV, lymphocyte count, WBC count, platelets, reticulocytes) and autoimmune testing (ANA, RF). Additional testing can be performed for specific populations who may be or have been exposed to other diseases such as TB, malaria, dengue. All of these tests can be performed for convenience and speed within about an hour at a retail health clinic such as an urgent care center or retail pharmacy for purposes of rapidly issuing a life insurance policy. Certain exemplary instruments and tests are currently CLIA waived and can run on just a drop of blood. Examples of such instruments include the ABX Pentra 60C and Sysmex POCH 100i for hematology and CBC. The IStat Chem 8 takes a metabolic panel measurement, the Cholestech LDx measure different lipids, liver enzymes and glucose, and the Piccolo POC Blood Chemistry Analyzer measures kidney, liver and metabolic indices.

In some embodiments, full panels of clinical chemistry and hematology tests can now be conducted in a retail pharmacy, urgent care clinic, retail clinics or retail health clinics without the need to send samples to central lab for testing. For example, hand-held clinical chemistry analyzer and hematology analyzer can be used for such tests. This new process eliminates time and assures more rapid underwriting for larger coverage policies. This new process also eliminates the need for testing and sampling at the applicant's home as all testing and sampling can be done at the retail pharmacy or urgent care clinic where the point of presence chemistry and hematology analyzers will be located. All lab information can be collated onto one instrument and/or computer and sent as a complete report to the underwriter from the retail or urgent care setting.

In some embodiments, the present kits and systems can comprise lateral flow devices and/or related instruments and/or systems disclosed and/or claimed in at least one of the following patents and applications: U.S. Pat. Nos. 5,073,484, 5,654,162, 6,020,147, 4,695,554, 4,703,017, 4,743,560, 5,591,645, RE 38,430 E, 5,602,040, 5,633,871, 5,656,503, 6,187,598, 6,228,660, 6,818,455, 7,109,042, 6,352,862, 7,238,537, 7,384,796, 7,407,813, 5,714,389, 5,989,921, 6,485,982, 6,485,982 C1, 5,120,643, 5,578,577, 6,534,320, 5,252,496, 5,559,041, 5,728,587, 6,027,943, 6,506,612, 6,541,277, 6,737,277, 7,175,992 B2, 7,691,595 B2, 6,770,487 B2, 7,247,500 B2, 7,662,643 B2, 5,712,170, 5,965,458, 7,371,582 B2, 7,476,549 B2, 7,633,620 B2, 7,815,853, 6,861,214 B1, 6,544,797 B1, 7,588,908 B2, 6,267,722 B1, 6,394,952 B1, 6,867,051 B1, 6,936,476 B1, 7,270,970 B2, 6,830,731 B1, 7,416,700 B1, 7,239,394 B2, 7,315,378 B2, 7,317,532 B2, 7,616,315 B2, 7,521,259 B2,521,260 B2, US 2005/0221504 A1, US 2005/0221505 A1, U.S. Pat. No. 8,128,871B2, US 2007/0143035 A1, US 2007/0185679 A1, U.S. Pat. No. 8,024,148, US 2009/0180925 A1, US 2009/0180926 A1, US 2009/0180927 A1, US 2009/0180928 A1, US 2009/0180929 A1, US 2009/0214383 A1, U.S. Pat. No. 6,777,198, US 2009/0311724 A1, US 2009/0117006 A1, U.S. Pat. Nos. 7,256,053, 6,916,666, 6,812,038, 5,710,005, 6,140,134, US 2010/0143941 A1, U.S. Pat. Nos. 6,140,048, 6,756,202, 7,205,553, 7,679,745, US 2010/0165338 A1, US 2010/0015611 A1, U.S. Pat. Nos. 7,178,416, 7,784,678 B2, 8,046,175 B2, US 2010/0173423 A1, US 2009/0157023 A1, U.S. Pat. Nos. 7,785,899 B2, 7,763,454 B2, US 2010/0239460 A1, US 2010/0240149 A1, U.S. Pat. Nos. 7,796,266 B2, 7,815,854 B2, US 2005/0244953 A1, US 2007/0121113 A1, US 2003/0119202 A1, US 2010/0311181 A1, 6,707,554 B1, U.S. Pat. Nos. 6,194,222 B1, 7,713,703, and 8,124,359 B2.

The present kits and systems can be used for assessing any suitable life insurance applications. Life insurance can include all forms of individual and group insurance coverage that contain life contingencies such as term life, whole life, endowment life, universal life, variable life, variable universal life, accidental death, life settlement, pension, and annuities. These contracts can provide for the payment of a beneficiary in the event of a person's death (e.g., term life insurance) or the payment of a beneficiary during the lifetime of an annuitant (e.g., an annuity or pension plan). Exemplary life insurance can include mortgage life insurance, permanent life insurance, term life insurance, universal life insurance (often shortened to UL), variable universal life insurance (often shortened to VUL) and whole life insurance, or whole of life assurance.

The present kits and systems can be used for assessing any suitable health insurance applications. Health insurance can include all forms of individual and group health insurance coverage that involve individual health contingencies such as major medical, HMO, long term care, disability income, limited benefit forms of coverage such as dental care, and accident coverage. These plans can provide either for the reimbursement of an insured for covered health care services (e.g., major medical plans) or the payment of a stated amount of money on a periodic basis in connection with a health contingency (e.g., disability income or long term care insurance). Exemplary health insurance can include accidental death and dismemberment (also known as AD&D), medical insurance, dental insurance, disability insurance (often called DI or disability income insurance), total permanent disability (TPD), and income protection insurance (IPI).

### C. Devices and Systems for Assessing Life or Health Insurance Applicants

In still another aspect, the present disclosure provides a lateral flow test device for assessing a life or health insurance applicant, which device is configured to assess at least two analytes in a sample derived from a life or health insurance applicant, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and said device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to assess said at least two analytes in a sample, and the formation of said detectable signal requires the use of a detectable label.

The present devices can be used for assessing a life or health insurance applicant at any suitable location. In some embodiments, the present devices can be used for assessing a life or health insurance applicant at a point of presence, e.g., at the location where a sample from an applicant is obtained, an insurance application is submitted and/or accepted, and/or an insurance decision is made. Exemplary point of presence can be a residential place, an office, e.g., a medical office or an insurance office, a retail location or a store, e.g., a pharmacy store or a supermarket, a clinical laboratory, or a health station or kiosk. Point of presence does not mean that the rapid tests must be conducted in the presence of a life or health insurance applicant. In some embodiments, the rapid tests can be conducted in the presence of a life or health insurance applicant. In other embodiments, the rapid tests can be conducted in the absence of a life or health insurance applicant. For example, after a sample is obtained from a life or health insurance applicant, the life or health insurance applicant may leave the location where the sample from the applicant is obtained before or during the rapid tests are conducted, and the applicant can be notified of the insurance decision subsequently.

The readout signal from tests using the present devices can be assessed by any suitable means. In some embodiments, the readout signal from tests using the present devices can be assessed by using any suitable label-free detection methods. Exemplary label-free detection methods include the detection methods based on surface plasmon resonance, bio-layer interferometry, epic technology, quartz crystal microbalance, electrical impedance and microcalorimetry. See e.g., Yu and White, “Optofluidic SERS on Paper: A Lateral Flow Concentration Assay Using Inkjet Fabricated SERS-Active Substrates,” in CLEO: Science and Innovations, OSA Technical Digest (online) (Optical Society of America, 2012), paper CTh4L.1; and Xiao et al., “A lateral flow biosensor for detection of single nucleotide polymorphism by circular strand displacement reaction,” Chem. Commun. (London), 48(68):8547-9 (2012). In some embodiments, the readout signal from tests using the present kits can be assessed by using any suitable detection methods using a detectable label.

The present device can be configured to assess any suitable number of analytes, e.g., 2, 3, 4, 5, 6, 7, 8, 9, 10, 11, 12, 13 or 14 analytes. In some embodiments, the present device is configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP. In other embodiments, the present device is configured to assess 5 analytes selected from the group consisting of an anti-HIV antibody, an anti-HCV antibody, cotinine and/or nicotine, cocaine and/or benzoylecgonine, and Hemoglobin A1c.

The present device can be used in any assay format e.g., a sandwich assay or a competitive assay format or both. In some embodiments, the present device is to be used in a sandwich assay for the analytes and wherein the test reagents at the test locations bind to the analytes, and a binding reagent that comprises a detectable label and binds to the analytes is used. In other embodiments, the present device is to be used in a sandwich assay for the analytes and wherein test reagents at the test locations bind to the analyte, and a binding reagent that comprises a detectable label and binds to another binding reagent that binds to the analyte is used. Any suitable number of binding reagent(s) can be used. In some embodiments, a single binding reagent that comprises a detectable label and binds to the analytes is used. In other embodiments, multiple binding reagents that comprise a detectable label and bind to the analytes are used. Any suitable type of binding reagent(s) can be used. For example, the binding reagent(s) can be an antibody or multiple antibodies. In some embodiments, the antibody or multiple antibodies specifically bind to the analytes.

In some embodiments, the present device is to be used in a competitive assay for the analytes and wherein the test reagents at the test locations are analytes or an analyte analogs, a second binding reagent that comprises a detectable label and binds to the analytes is used, and the analytes or an analyte analogs at the test locations compete with analytes in the sample for binding to the second binding reagent. In other embodiments, the present device is to be used in a competitive assay for the analytes and wherein the test reagents at the test locations are analytes or an analyte analogs, a second binding reagent that comprises a detectable label and binds to another binding reagent that binds to the analytes is used, and the analytes or an analyte analogs at the test locations compete with the analytes in the sample for binding to the binding reagent that is bound to the second binding reagent. In still other embodiments, the rapid test devices, e.g., lateral flow test devices, can be used in a competitive assay for the analytes and wherein the test reagents at the test locations are binding reagents that bind to the analytes, analytes or analyte analogs linked to a detectable label are used, and the analytes or analyte analogs linked to a detectable label compete with analytes in the sample for binding to the binding reagents at the test locations. Any suitable number of binding reagent(s) can be used. In some embodiments, a single second binding reagent is used. In other embodiments, multiple second binding reagents are used. Any suitable type of binding reagent(s) can be used. For example, the binding reagent(s) can be an antibody or multiple antibodies. In some embodiments, the antibody or multiple antibodies specifically bind to the analytes.

The present devices can be used to assess any suitable analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP. In some embodiments, the analytes are polypeptides, polynucleotides or small molecules, and the test reagents and/or binding reagents that bind to the analytes are antibodies that bind to the polypeptides or small molecules. In some embodiments, the analytes are polypeptides or small molecules, and the test reagent and/or binding reagent that binds to the analytes are binding reagents for the analytes. Preferably, the binding reagents bind specifically to the analytes. Any suitable binding reagents can be used. In some embodiments, the binding reagent can be an antibody that binds to the polypeptide or small molecule. Preferably, the antibody binds specifically to the analyte. In some embodiments, the analyte are polynucleotides, and the binding reagents are other polynucleotides that are complementary or substantially complementary with the analyte polynucleotides.

The matrix can comprise any suitable material(s). In some embodiments, the matrix comprises nitrocellulose, glass fiber, polypropylene, polyethylene (preferably of very high molecular weight), polyvinylidene fluoride, ethylene vinyl acetate, acrylonitrile and/or polytetrafluoro-ethylene.

The matrix can be in any suitable shape. In some embodiments, the matrix is in the form a strip or a circle. The matrix can comprise any suitable number of element(s). For example, the matrix can be a single element or can comprise multiple elements.

In some embodiments, the present devices can further comprise a sample application element upstream from and in fluid communication with the matrix. In other embodiments, the present devices can further comprise a liquid absorption element downstream from and in fluid communication with the matrix. In still other embodiments, the present devices can further comprise a control zone comprising means for indicating proper flow of the liquid sample and/or a valid test result. In yet other embodiments, at least a portion of the matrix is supported by a solid backing.

In some embodiments, a substance can be dried on a portion of the matrix upstream from the test locations, the analytes and/or dried substance being capable of being moved by a liquid sample and/or a further liquid to the test locations and/or a control location to generate a detectable signal, the dried substance being at least one of the second binding reagent that binds to the analyte, the second binding reagent that binds to another binding reagent that binds to the analyte, the analyte or the analyte analog, each of the second binding reagent, analyte or analyte analog comprises a detectable label.

Any suitable number of substance(s) can be used. In some embodiments, a single substance is dried on a portion of the matrix upstream from the test locations. In other embodiments, multiple substances are dried on a portion of the matrix upstream from the test locations. The substance can be located at any suitable locations. In some embodiments, the substance can be dried on a conjugate element that is upstream from the test zone. In other embodiments, the substance can be located downstream from a sample application place on the test device. In still some embodiments, the substance can be located upstream from a sample application place on the test device.

Any suitable detectable label can be used. In some embodiments, the detectable label can be a soluble label, e.g., a soluble enzyme or fluorescent label. In other embodiments, the detectable label can be a particle label. For example, the particle label can be a visible or a non-visible particle label. In some embodiments, the visible particle label can be a colloidal gold label, a latex particle label, a nanoparticle label or a quantum dot label. In some embodiments, the non-visible particle label can be a fluorescent particle.

In some embodiments, the substance can be dried in the presence of a material that: a) stabilizes the dried substance; b) facilitates solubilization or re-suspension of the dried substance in a liquid; and/or c) facilitates mobility of the dried substance. Any suitable substance can be used. In some embodiments, the material can be a protein, a peptide, a polynucleotide, a polysaccharide, a sugar, a polymer, a gelatin or a detergent.

The analytes and/or the substance(s) can be transported to the test locations using any suitable liquid. In some embodiments, a sample liquid alone is used to transport the analytes and/or the substance to the test locations. In other embodiments, a developing liquid is used to transport the analytes and/or the substance to the test locations.

The present device can further comprise a housing that covers at least a portion of the test device, wherein the housing comprises a sample application port to allow sample application upstream from or to the test locations and an optic opening around the test locations to allow signal detection at the test locations. In some embodiments, the housing covers the entire test device. In other embodiments, at least a portion of the sample receiving portion of the matrix or the sample application element is not covered by the housing and a sample is applied to the portion of the sample receiving portion of the matrix or the sample application element outside the housing and then transported to the test locations.

The present device can be configured to receive any suitable types of samples. In some embodiments, the present device is configured to receive at least two different types of samples, e.g., a blood sample and a saliva sample.

The test locations in the present device can be placed at any suitable locations or pathways. In some embodiments, the test locations are in the same liquid flow pathway. In other embodiments, the test locations are in the different liquid flow pathways. Preferably, the different liquid flow pathways are substantially parallel to each other and are shielded from each other.

The present device can be configured to assess at least one of the analytes qualitatively, quantitatively or semi-quantitatively. In some embodiments, the present device can be configured to assess 2, 3, 4, 5, 6, 7, 8, 9, 10, 11, 12, 13, or 14 analytes qualitatively, quantitatively or semi-quantitatively.

In yet another aspect, the present disclosure provides for a system for assessing a life or health insurance applicant, which system comprises any of the above lateral flow test devices.

In some embodiments, the present system can further comprise an instruction for using the system for assessing mortality and/or morbidity risk of the life or health insurance applicant, and/or making a decision on the insurance or health application from the life insurance applicant.

In some embodiments, the present system can further comprise a means for assessing an additional health indicator of the life or health insurance applicant. Any suitable means can be used. For example, the means can be used to assess a blood pressure, a body mass index and/or alcohol consumption or alcoholism of the life or health insurance applicant. Any suitable means for detecting alcohol consumption or alcoholism can be used. For example, a number of questionnaires that are used to detect or diagnosis alcoholism can be included in the present system. In another example, test devices and/or reagents for detecting alcohol consumption or alcoholism, such as test devices and/or reagents for detecting gammaglutamyl transferase (ggt) and other liver function tests, mean corpuscular volume (MCV), folate/b12, magnesium, or carbohydrate deficient form of transferrin (CDT), can be included in the present system.

In some embodiments, the present system can further comprise a means for assessing an identifier of the life or health insurance applicant. Any suitable means can be used. For example, the means can be an iris scan, a fingerprinting device, a haplotyping device, or a nucleic acid, e.g., DNA sequencer, or a means for assessing voice identification, photograph, electronic signature, or photo identification. In some embodiments, the nucleic acid, e.g., DNA, sequence information from an applicant can be used to verify the identity of the applicant. In some embodiments, the nucleic acid, e.g., DNA, sequence information from an applicant is not used or considered in the insurance underwriting.

In some embodiments, the present system can further comprise a means for obtaining at least two different types of samples from the life or health insurance applicant. In other embodiments, the present system can further comprise a means for collecting more than one finger stick of blood from the life or health insurance applicant to increase the volume of blood sample to be able to run multiple assays on the blood sample. In still other embodiments, the present system can further comprise a finger stick device that simultaneously makes at least two punctures at once to increase the amount of blood sample volume needed to perform multiple assays on the blood sample.

The present system can further comprise machine-readable information, e.g., a bar code. The machine-readable information can be located at any suitable locations, e.g., as part of the lateral flow device(s) or as a separate component on the system. The machine-readable information can be comprised in any suitable storage medium, e.g., a RFID device.

The present system can further comprise a reader to assess the detectable signal. Any suitable reader can be used, e.g., a fluorescent reader, a colorimetric reader, or luminescent reader. Any fluorescent suitable reader can be used, e.g., a laser based or a light emitting diode (LED) based fluorescent reader. The present system can further comprise a means for recording, storing and/or sending test results in an electronic format, and/or a software program and/or algorithm that collates and/or optimizes all data inputs such as assay results, BMI, application, release form, attending physician statement (APS), medical information bureau (MIB) record, motor vehicle record (MVR), credit report and prescription or transaction history record into one electronically transferrable or printable file, and/or an apparatus, software and/or algorism for forensic voice analysis that detects emotions in the human voice. Any suitable apparatus, software or algorism for forensic voice analysis that detects emotions in the human voice can be used. See e.g., U.S. Pat. No. 7,165,033.

The present kits and systems can be used for assessing any suitable life insurance applications. Life insurance can include all forms of individual and group insurance coverage that contain life contingencies such as term life, whole life, endowment life, universal life, variable life, variable universal life, accidental death, life settlement, pension, and annuities. These contracts can provide for the payment of a beneficiary in the event of a person's death (e.g., term life insurance) or the payment of a beneficiary during the lifetime of an annuitant (e.g., an annuity or pension plan). Exemplary life insurance can include mortgage life insurance, permanent life insurance, term life insurance, universal life insurance (often shortened to UL), variable universal life insurance (often shortened to VUL) and whole life insurance, or whole of life assurance.

The present devices and systems can be used for assessing any suitable health insurance applications. Health insurance can include all forms of individual and group health insurance coverage that involve individual health contingencies such as major medical, HMO, long term care, disability income, limited benefit forms of coverage such as dental care, and accident coverage. These plans can provide either for the reimbursement of an insured for covered health care services (e.g., major medical plans) or the payment of a stated amount of money on a periodic basis in connection with a health contingency (e.g., disability income or long term care insurance). Exemplary health insurance can include accidental death and dismemberment (also known as AD&D), medical insurance, dental insurance, disability insurance (often called DI or disability income insurance), total permanent disability (TPD), and income protection insurance (IPI).

The present devices and/or systems can further comprise a solid medium for collecting, drying and/or storing a sample derived from a life or health insurance applicant wherein said collected, dried and/or stored sample can be used in a test.

Any suitable solid medium can be used in the present devices and/or systems. In some embodiments, the solid medium is a porous solid medium. Any suitable porous solid medium can be used. For example, the porous solid medium can comprise filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene. In other embodiments, the solid medium is a non-porous solid medium. Any suitable non-porous solid medium can be used. For example, the non-porous solid medium can comprise a plastic material or glass slide.

In some embodiments, the solid medium can be contained in a container or device can be at least a part of a surface of a container. Any suitable container can be used. For example, the container can be a tube or a microtiter plate.

Any suitable sample derived from a life or health insurance applicant can be collected, dried and/or stored on or in the solid medium of the present devices and/or systems. In some embodiments, the dried sample is a dried body fluid sample, e.g., a whole blood, a serum, a plasma, a fresh blood, a blood not containing an anti-coagulate, a urine and a saliva sample. In some embodiments, the dried sample comprises an analyte, e.g., a polypeptide, a polynucleotide or a small molecule.

In some embodiments, the sample can be collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content. Any such suitable material can be used. For example, the material can be a protein, a peptide, a polynucleotide, a polysaccharide, a sugar, a polymer, a gelatin and a detergent.

In some embodiments, the present disclosure provides for devices and/or systems wherein the solid medium comprises a collected, dried and/or stored sample.

The sample can be collected, dried and/or stored on or in a solid medium in any suitable manner or form. For example, the sample can be dried on a solid medium as a dried spot.

The sample can be stored and remain useable and/or stable for any suitable period of time. For example, the sample can be stored for a period of time during which the insurance application and/or decision can be reviewed, reassessed and/or contested. In some embodiments, the dried sample can be stored for a short term, e.g., about 1, 2, 3, 4, 5, 6, 7, 8, 9 or 10 days. In other embodiments, the dried sample can be stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

The collected, dried and/or stored sample can be used for any suitable purpose. In some embodiments, the collected, dried and/or stored sample can be retested for an analyte that has been tested before for assessing a life or health insurance applicant from which the sample is derived. In other embodiments, the collected, dried and/or stored sample can be tested for an analyte that has not been tested before. In still other embodiments, the collected, dried and/or stored sample can be tested for confirmation of the presence or absence or concentration of an analyte. In yet other embodiments, the collected, dried and/or stored sample can be tested for assessing an identifier, e.g., a personal identifier, of the life or health insurance applicant.

In some embodiments, in the event of a positive result with the rapid insurance tests that most likely may be a public health issue or disputed issue by the applicant, a sample at the time of collection can be saved through a dried body fluid spot that can be used to re-test the applicant or patient or without calling he/she back to the retail pharmacy or urgent care clinic to re-sample. The dried spot sample can be re-tested immediately, sent for further analysis such as confirmation or saved for applicant identification.

In some embodiments, a sample at the time of collection can be saved through a dried body fluid spot using any suitable devices and methods know in the art. Such exemplary devices and methods include methods of drying blood for stable glucose testing disclosed in U.S. Pat. No. 5,204,267; an apparatus for preparing and processing dried blood spots for HIV testing on a test card and punching out an exact area that represents a quantifiable amount of blood residue disclosed in U.S. Pat. Nos. 5,641,682, 5,862,729 and 6,171,868; devices and methods for preserving and extracting nucleic acids from cells from whole blood dried blood spots for nucleic acid testing disclosed in U.S. Pat. No. 7,927,798; devices and methods for quantitative recovery of physiologically important enzymes from body fluids including dried blood and saliva spots disclosed in U.S. Pat. No. 6,756,230; and an apparatus for collecting and drying body fluid samples for later analysis disclosed in U.S. Pat. No. 7,638,099.

The present systems can further comprise a means for collecting additional sample from a life or health insurance applicant to be stored on a solid medium. Any suitable means can be used. For example, the means can comprise a device for collecting a sample to be dried or stabilized for storage purpose. The dried or stabilized sample can be used for any suitable purposes. For example, the dried or stabilized sample can be suitable for an optional test that can be completed at a later time, e.g., an optional test for assessing optional insurance coverage.

The system can further comprise a device or instrument for conducting a thyroid test, a thyroid panel test, a test for liver marker, a metabolic panel test, a lipid test, a test for blood count, an autoimmune test, and/or an infectious disease test on a sample from a life or health insurance applicant.

The present systems can comprise any suitable thyroid test. For example, the present systems can comprise a thyroid test that assesses thyroid function using thyroid-stimulating hormone (TSH). The present systems can comprise any suitable thyroid panel test. For example, the present systems can comprise a thyroid panel test that assesses TSH, thyroxine (T4), and Triiodothyronine (T3).

The present systems can comprise any suitable test for liver function. For example, the present systems can comprise a test for liver disease by measuring Gamma-glutamyltransferase (GGT), antinuclear antibodies (ANA), alkaline phosphatase (ALP), albumin, and/or prothrombin.

The present systems can comprise any suitable metabolic panel test. For example, the present systems can comprise a metabolic panel test that assesses acid/base balance, glucose, sodium, potassium, blood urea nitrogen (BUN), creatinine, and/or blood gases.

The present systems can comprise any suitable lipid test. For example, the present systems can comprise a lipid test that assesses apolipoprotein A-I (ApoA1) and/or apolipoprotein B (ApoB).

The present systems can comprise any suitable test for blood diseases. For example, the present systems can comprise a complete blood count that comprises hematocrit, hemoglobin, mean corpuscular volume (MCV), lymphocyte count, white blood cell (WBC) count, platelets, and reticulocytes.

The present systems can comprise any suitable autoimmune test. For example, the present systems can comprise an autoimmune test that assesses ANA and/or rheumatoid factor (RF).

The present systems can comprise any suitable infectious disease test. For example, the present systems can comprise an infectious disease test that assesses a maker for tuberculosis (TB), malaria, and/or dengue.

The present systems can comprise any suitable device or instrument for conducting a thyroid test, a thyroid panel test, a test for a liver marker, a metabolic panel test, a lipid test, a test for blood count, an autoimmune test, and/or an infectious disease test on a sample from a life or health insurance applicant. For example, the device or instrument can be a clinical chemistry analyzer, a hematology analyzer, or a hand-held device or instrument. The device or instrument can conduct a test within any suitable period of time. For example, the device or instrument can conduct a test within about 1 hour, 2 hours, 4 hours, 8 hours, 16 hours, or 24 hours.

In some embodiments, further testing can be undertaken at the retail clinic, retail pharmacy such as Walgreens, or urgent care clinic, and can be accomplished in under one-hour time. Such exemplary testing can include a thyroid test (TSH) or thyroid panels (TSH, T4, T3), additional liver testing (GGT, ANA, ALP, albumin, prothrombin), metabolic panels (acid/base balance, glucose, sodium, potassium, BUN, creatinine, blood gases), additional lipid tests (ApoA1, ApoB), and complete blood count (hematocrit, hemoglobin, MCV, lymphocyte count, WBC count, platelets, reticulocytes) and autoimmune testing (ANA, RF). Additional testing can be performed for specific populations who may be or have been exposed to other diseases such as TB, malaria, dengue. All of these tests can be performed for convenience and speed within about an hour at a retail health clinic such as an urgent care center or retail pharmacy for purposes of rapidly issuing a life insurance policy. Certain exemplary instruments and tests are currently CLIA waived and can run on just a drop of blood. Examples of such instruments include the ABX Pentra 60C and Sysmex POCH 100i for hematology and CBC. The IStat Chem 8 takes a metabolic panel measurement, the Cholestech LDx measure different lipids, liver enzymes and glucose, and the Piccolo POC Blood Chemistry Analyzer measures kidney, liver and metabolic indices.

In some embodiments, full panels of clinical chemistry and hematology tests can now be conducted in a retail pharmacy, urgent care clinic, retail clinics or retail health clinics without the need to send samples to central lab for testing. For example, hand-held clinical chemistry analyzer and hematology analyzer can be used for such tests. This new process eliminates time and assures more rapid underwriting for larger coverage policies. This new process also eliminates the need for testing and sampling at the applicant's home as all testing and sampling can be done at the retail pharmacy or urgent care clinic where the point of presence chemistry and hematology analyzers will be located. All lab information can be collated onto one instrument and/or computer and sent as a complete report to the underwriter from the retail or urgent care setting.

### D. Methods for Assessing Life or Health Insurance Applicants

In yet another aspect, the present disclosure provides a method for assessing a life or health insurance applicant, which method comprises: a) assessing the presence, absence and/or amount of at least two analytes in a sample derived from a life or health insurance applicant using a rapid test, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and b) assessing an insurance application from said life or health insurance applicant based on the test results obtained in step a).

The present methods can be used for assessing a life or health insurance applicant at any suitable location. In some embodiments, the present methods can be used for assessing a life or health insurance applicant at a point of presence, e.g., at the location where a sample from an applicant is obtained, an insurance application is submitted and/or accepted, and/or an insurance decision is made. Exemplary point of presence can be a residential place, an office, e.g., a medical office or an insurance office, a retail location or a store, e.g., a pharmacy store or a supermarket, a clinical laboratory, or a health station or kiosk. Point of presence does not mean that the rapid tests must be conducted in the presence of a life or health insurance applicant. In some embodiments, the rapid tests can be conducted in the presence of a life or health insurance applicant. In other embodiments, the rapid tests can be conducted in the absence of a life or health insurance applicant. For example, after a sample is obtained from a life or health insurance applicant, the life or health insurance applicant may leave the location where the sample from the applicant is obtained before or during the rapid tests are conducted, and the applicant can be notified of the insurance decision subsequently.

Any suitable rapid tests can be used in the present methods. For example, the kits and systems described in the above Section VI.B, and the devices and systems described in the above Section VI.C can be used.

In some embodiments, the kits and systems described in the above Section VI.B, specially the kits and systems comprising lateral flow devices, can be used, which method comprises: a) contacting a liquid sample with at least one of the rapid test devices, e.g., lateral flow test devices, in the kits and systems described in the above Section VI.B, wherein the liquid sample is applied to a site of the test device upstream of the test zone; b) transporting an analyte, if present in the liquid sample, and a labeled reagent to the test zone; c) assessing a detectable signal at the test zone to determine the presence, absence and/or amount of the analyte; and wherein steps a)-c) are conducted using at least two of the rapid test devices, e.g., lateral flow test devices, in the kits and systems described in the above Section VI.B to assess the presence, absence and/or amount of at least two analytes in the sample.

In other embodiments, the devices and systems described in the above Section VI.C can be used, which method comprises: a) contacting a liquid sample with the device or system described in the above Section VI.C, wherein the liquid sample is applied to a site of the test device upstream of the test locations; b) transporting multiple analytes, if present in the liquid sample, and a labeled reagent to the test locations; and c) assessing a detectable signal at the test locations to determine the presence, absence and/or amount of the analyte in the sample.

A labeled reagent is used to generate a detectable signal in the present methods. The labeled reagent can be used in any suitable manner. In some embodiments, a liquid sample and a labeled reagent can be premixed to form a mixture and the mixture is applied to the test device. The present methods can further comprise a washing step after the mixture is applied to the test device. The washing step can be conducted in any suitable manner. For example, the test device can comprise a liquid container comprising a washing liquid and the washing step can comprise releasing the washing liquid from the liquid container. See e.g., U.S. Pat. No. 5,137,808.

In other embodiments, the test device can comprise a dried labeled reagent before use and the dried labeled reagent can be solubilized or re-suspended, and transported to the test zone or locations by a liquid. The labeled reagent can be placed at any suitable location of the device. For example, the dried labeled reagent can be located downstream from the sample application site, and the dried labeled reagent can be solubilized or re-suspended, and transported to the test zone or location by the liquid sample. In another example, the dried labeled reagent can be located upstream from the sample application site, and the dried labeled reagent can be solubilized or re-suspended, and transported to the test zone or locations by another liquid.

The labeled reagent can be solubilized or re-suspended, and transported to the test zone or location by any suitable liquid. In some embodiments, the labeled reagent is solubilized or re-suspended, and transported to the test zone or location by the liquid sample alone. In other embodiments, the analyte(s) and/or labeled reagent are solubilized or re-suspended, and transported to the test zone or location by another liquid.

Any suitable sample can be used in the present methods. In some embodiments, the liquid sample is a body fluid sample. Any suitable body fluid sample can be used. For example, the body fluid sample can be a whole blood, a serum, a plasma, a fresh blood, a blood not containing an anti-coagulate, a urine or a saliva sample.

Generally, suitable sample can be selected based on the analyte(s) to be tested. For example, a liver enzyme alanine transaminase (ALT), a liver enzyme aspartate transaminase (AST), nt-probnp (or NT-proBNP), probnp (or proBNP), ApoA1, ApoB, Hemoglobin A1c or hsCRP can be assessed using a blood sample. In another example, a HIV antigen (e.g., a HIV polypeptide), a HIV polynucleotide, an anti-HIV antibody, a HCV antigen (e.g., a HCV polypeptide), a HCV polynucleotide, an anti-HCV antibody, cotinine, nicotine, cocaine or benzoylecgonine can be assessed using a saliva sample. Any suitable HIV antigen (e.g., a HIV polypeptide) or HIV polynucleotide can be detected. For example, a HIV antigen (e.g., a HIV polypeptide) or HIV polynucleotide from HIV-1, HIV-2 or both can be detected. Any suitable antibody to a HIV antigen can be detected. For example, an antibody to a HIV-1 antigen, a HIV-2 antigen or both can be detected. In some embodiments, an antibody to Group 0 antigen from HIV-1, HIV-2 or both can be detected. The rapid tests can be based on any suitable test principle such as affinity chromatography. For example, Hemoglobin A1c can be detected using boronate affinity or immuno affinity methods.

Any suitable number of analytes, e.g., 2, 3, 4, 5, 6, 7, 8, 9, 10, 11, 12, 13, or 14 analytes, can be assessed in the present methods. In some embodiments, at least 3, 4, or 5 analytes are assessed. In other embodiments, the present methods comprises assessing 5 analytes selected from the group consisting of an anti-HIV antibody, an anti-HCV antibody, cotinine and/or nicotine, cocaine and/or benzoylecgonine, and Hemoglobin A1c.

Any suitable assay format can be used in the present methods. For example, an anti-HIV antibody, an anti-HCV antibody, and Hemoglobin A1c can be assessed using a sandwich assay. In another example, cotinine and/or nicotine, and cocaine and/or benzoylecgonine can be assessed using a competitive assay.

In some embodiments, the present methods can be used to assess mortality and/or morbidity of a life or health insurance applicant.

The present methods can be used to assess analyte(s) qualitatively, quantitatively or semi-quantitatively. In some embodiments, at least one of the analytes can be assessed qualitatively. For example, 2, 3, 4, 5, 6, 7, 8, 9, 10, 11, 12, 13, or 14 analytes can be assessed qualitatively. In other embodiments, at least one of the analytes can be assessed quantitatively or semi-quantitatively. For example, 2, 3, 4, 5, 6, 7, 8, 9, 10, 11, 12, 13, or 14 analytes can be assessed quantitatively or semi-quantitatively. In some embodiments, the analytes can be assessed similarly, e.g., all the analytes are assessed qualitatively, quantitatively or semi-quantitatively. In other embodiments, the analytes can be assessed differentially, e.g., some analytes are assessed qualitatively and other analytes are assessed quantitatively or semi-quantitatively.

The readout signal from the present methods can be assessed by any suitable means. In some embodiments, the readout signal from the present methods can be assessed by using any suitable label-free detection methods. Exemplary label-free detection methods include the detection methods based on surface plasmon resonance, bio-layer interferometry, epic technology, quartz crystal microbalance, electrical impedance and microcalorimetry. The label-free detection methods disclosed and/or claimed in the following U.S. Pat. Nos. can also be used: U.S. Pat. Nos. 7,531,786, 7,737,392, 7,742,662, 7,790,406, 7,863,052, 7,955,883, 7,960,170, 7,968,836 and 8,145,434. In some embodiments, the readout signal from the present methods can be assessed by using any suitable detection methods using a detectable label.

The detectable signal can be assessed in any suitable manner, e.g., with or without using a reader. Any suitable reader can be used. Exemplary readers include a fluorescent reader, a colorimetric reader, and a luminescent reader. In some embodiments, a fluorescent signal is generated and the fluorescent signal is assessed by a fluorescent reader. Any suitable fluorescent reader can be used. For example, the fluorescent reader is a laser based or a light emitting diode (LED) based fluorescent reader. The present methods can further comprise a step for recording, storing and/or sending test results in an electronic format, and/or using a software program and/or algorithm to collate and/or optimize all data inputs such as assay results, BMI, application, release form, attending physician statement (APS), medical information bureau (MIB) record, motor vehicle record (MVR), credit report and prescription or transaction history record into one electronically transferrable or printable file, and/or for conducting forensic voice analysis of the voice of an applicant. Any suitable apparatus, software or algorism for forensic voice analysis that detects emotions in the human voice can be used. See e.g., U.S. Pat. No. 7,165,033.

A sample can be derived from a life or health insurance applicant in any suitable manner. For example, a sample can be derived from a life or health insurance applicant with fasting (e.g., food fasting). Fasting is primarily the act of willingly abstaining from all food, drink, or both, for a period of time. Any suitable fasting period can be used, e.g., several days, a single day, 16 hours, 12 hours, 8 hours, 4 hours, 2 hours or less time. In another example, a sample can be derived from a life or health insurance applicant without fasting (e.g., food fasting).

The present methods can be conducted within any suitable time frame. For example, from the time a sample is taken from an applicant, the present methods can be conducted in about 12, 11, 10, 9, 8, 7, 6, 5, 4, 3, 2 or 1 hour, 50 minutes, 40 minutes, 30 minutes, 20 minutes, or 10 minutes to support the assessment of an insurance application from the life or health insurance applicant based on the test results.

The present methods can further comprise assessing an additional health indicator of a life insurance or health applicant. Exemplary health indicators include a blood pressure, a body mass index and/or alcohol consumption or alcoholism of the life insurance or health applicant. Any suitable methods for detecting alcohol consumption or alcoholism can be used. For example, a number of questionnaires that are used to detect or diagnosis alcoholism can be used in the present methods. In another example, test devices and/or reagents for detecting alcohol consumption or alcoholism, such as test devices and/or reagents for detecting gammaglutamyl transferase (ggt) and other liver function tests, mean corpuscular volume (MCV), folate/b12, magnesium, or carbohydrate deficient form of transferrin (CDT), can be used in the present methods. The body mass index (BMI), or Quetelet index, is a measure for human body shape based on an individual's weight and height. See e.g., BMI Calssification, Global Database on Body Mass Index. WHO. 2006; and Eknoyan, Garabed “Adolphe Quetelet (1796-1874)—the average man and indices of obesity,” Nephrology Dialysis Transplantation 23 (1): 47-51 (2007). Body mass index is typically defined as the individual's body mass divided by the square of their height. The formulae often used in medicine produce a unit of measure of kg/m². BMI can also be determined using a BMI chart, which displays BMI as a function of weight (horizontal axis) and height (vertical axis) using contour lines for different values of BMI or colors for different BMI categories. See e.g., The Body Mass Index Table from the National Institutes of Health's NHLBI. Exemplary formulae for measuring BMI is also shown below:

$$\begin{matrix}BMI = \frac{mass ( kg )}{{( height ( m ) )}^{2}} \\ = \frac{mass ( lb )}{{( height ( in ) )}^{2}} \times {703}_{†}\end{matrix}$$

The present methods can further comprise assessing an identifier of a life or health insurance applicant. Any suitable identifier can be assessed. For example, an identifier of a life insurance or health applicant can be assessed by iris scanning, fingerprinting, haplotyping, nucleic acid, e.g., DNA sequencing, or by assessing voice identification, photograph, electronic signature, or photo identification of the applicant. In some embodiments, the nucleic acid, e.g., DNA, sequence information from an applicant can be used to verify the identity of the applicant. In some embodiments, the nucleic acid, e.g., DNA, sequence information from an applicant is not used or considered in the insurance underwriting.

In some embodiments, the present methods can use lateral flow devices and/or related instruments and/or systems disclosed and/or claimed in at least one of the following patents and applications: U.S. Pat. Nos. 5,073,484, 5,654,162, 6,020,147, 4,695,554, 4,703,017, 4,743,560, 5,591,645, RE 38,430 E, 5,602,040, 5,633,871, 5,656,503, 6,187,598, 6,228,660, 6,818,455, 7,109,042, 6,352,862, 7,238,537, 7,384,796, 7,407,813, 5,714,389, 5,989,921, 6,485,982, 6,485,982 C1, U.S. Pat. Nos. 5,120,643, 5,578,577, 6,534,320, 5,252,496, 5,559,041, 5,728,587, 6,027,943, 6,506,612, 6,541,277, 6,737,277, 7,175,992 B2, 7,691,595 B2, 6,770,487 B2, 7,247,500 B2, 7,662,643 B2, 5,712,170, 5,965,458, 7,371,582 B2, 7,476,549 B2, 7,633,620 B2, 7,815,853, 6,861,214 B1, 6,544,797 B1, 7,588,908 B2, 6,267,722 B1, 6,394,952 B1, 6,867,051 B1, 6,936,476 B1, 7,270,970 B2, 6,830,731 B1, 7,416,700 B1, 7,239,394 B2, 7,315,378 B2, 7,317,532 B2, 7,616,315 B2, 7,521,259 B2,521,260 B2, US 2005/0221504 A1, US 2005/0221505 A1, U.S. Pat. No. 8,128,871B2, US 2007/0143035 A1, US 2007/0185679 A1, U.S. Pat. No. 8,024,148, US 2009/0180925 A1, US 2009/0180926 A1, US 2009/0180927 A1, US 2009/0180928 A1, US 2009/0180929 A1, US 2009/0214383 A1, U.S. Pat. No. 6,777,198, US 2009/0311724 A1, US 2009/0117006 A1, U.S. Pat. Nos. 7,256,053, 6,916,666, 6,812,038, 5,710,005, 6,140,134, US 2010/0143941 A1, 6,140,048, 6,756,202, 7,205,553, 7,679,745, US 2010/0165338 A1, US 2010/0015611 A1, U.S. Pat. Nos. 7,178,416, 7,784,678 B2, 8,046,175 B2, US 2010/0173423 A1, US 2009/0157023 A1, U.S. Pat. Nos. 7,785,899 B2, 7,763,454 B2, US 2010/0239460 A1, US 2010/0240149 A1, U.S. Pat. Nos. 7,796,266 B2, 7,815,854 B2, US 2005/0244953 A1, US 2007/0121113 A1, US 2003/0119202 A1, US 2010/0311181 A1, U.S. Pat. Nos. 6,707,554 B1, 6,194,222 B1, 7,713,703, and 8,124,359 B2.

The present methods can further comprise collecting, drying and/or storing a sample derived from a life or health insurance applicant on or in a solid medium wherein said collected, dried and/or stored sample can be used in a test.

Any suitable solid medium can be used in the present methods. In some embodiments, the solid medium is a porous solid medium. Any suitable porous solid medium can be used. For example, the porous solid medium can comprise filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene. In other embodiments, the solid medium is a non-porous solid medium. Any suitable non-porous solid medium can be used. For example, the non-porous solid medium can comprise a plastic material or glass slide

In some embodiments, the solid medium can be contained in a container or device can be at least a part of a surface of a container. Any suitable container can be used. For example, the container can be a tube or a microtiter plate.

Any suitable sample derived from a life or health insurance applicant can be collected, dried and/or stored on or in the solid medium for the present methods. In some embodiments, the dried sample is a dried body fluid sample, e.g., a whole blood, a serum, a plasma, a fresh blood, a blood not containing an anti-coagulate, a urine and a saliva sample. In some embodiments, the dried sample comprises an analyte, e.g., a polypeptide, a polynucleotide or a small molecule.

In some embodiments, the sample can be collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content. Any such suitable material can be used. For example, the material can be a protein, a peptide, a polynucleotide, a polysaccharide, a sugar, a polymer, a gelatin and a detergent.

The sample can be collected, dried and/or stored on or in a solid medium in any suitable manner or form. For example, the sample can be dried on a solid medium as a dried spot.

The sample can be stored and remain useable and/or stable for any suitable period of time. For example, the sample can be stored for a period of time during which the insurance application and/or decision can be reviewed, reassessed and/or contested. In some embodiments, the dried sample can be stored for a short term, e.g., about 1, 2, 3, 4, 5, 6, 7, 8, 9 or 10 days. In other embodiments, the dried sample can be stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

The collected, dried and/or stored sample can be used for any suitable purpose. In some embodiments, the collected, dried and/or stored sample can be retested for an analyte that has been tested before for assessing a life or health insurance applicant from which the sample is derived. In other embodiments, the collected, dried and/or stored sample can be tested for an analyte that has not been tested before. In still other embodiments, the collected, dried and/or stored sample can be tested for confirmation of the presence or absence or concentration of an analyte. In yet other embodiments, the collected, dried and/or stored sample can be tested for assessing an identifier, e.g., a personal identifier, of the life or health insurance applicant.

The present methods can further comprise collecting additional sample from a life or health insurance applicant to be stored on a solid medium. The additional sample can be collected by any suitable means. For example, the additional sample can be collected using a device and the collected sample can be dried or stabilized for storage purpose. The dried or stabilized sample can be used for any suitable purposes. For example, the dried or stabilized sample can be further tested, e.g., for assessing optional insurance coverage.

In some embodiments, in the event of a positive result with the rapid insurance tests that most likely may be a public health issue or disputed issue by the applicant, a sample at the time of collection can be saved through a dried body fluid spot that can be used to re-test the applicant or patient or without calling he/she back to the retail pharmacy or urgent care clinic to re-sample. The dried spot sample can be re-tested immediately, sent for further analysis such as confirmation or saved for applicant identification.

In some embodiments, a sample at the time of collection can be saved through a dried body fluid spot using any suitable devices and methods know in the art. Such exemplary devices and methods include methods of drying blood for stable glucose testing disclosed in U.S. Pat. No. 5,204,267; an apparatus for preparing and processing dried blood spots for HIV testing on a test card and punching out an exact area that represents a quantifiable amount of blood residue disclosed in U.S. Pat. Nos. 5,641,682, 5,862,729 and 6,171,868; devices and methods for preserving and extracting nucleic acids from cells from whole blood dried blood spots for nucleic acid testing disclosed in U.S. Pat. No. 7,927,798; devices and methods for quantitative recovery of physiologically important enzymes from body fluids including dried blood and saliva spots disclosed in U.S. Pat. No. 6,756,230; and an apparatus for collecting and drying body fluid samples for later analysis disclosed in U.S. Pat. No. 7,638,099.

The present methods can further comprise conducting a thyroid test, a thyroid panel test, a test for a liver marker, a metabolic panel test, a lipid test, a test for blood count, an autoimmune test, and/or an infectious disease test on a sample from a life or health insurance applicant.

The present methods can comprise conducting any suitable thyroid test. For example, the present methods can comprise conducting a thyroid test that assesses thyroid function using thyroid-stimulating hormone (TSH). The present methods can comprise conducting any suitable thyroid panel test. For example, the present methods can comprise conducting a thyroid panel test that assesses TSH, T4, and T3.

The present methods can comprise conducting any suitable test for liver function. For example, the present methods can comprise conducting a test for liver disease by measuring Gamma-glutamyltransferase (GGT), antinuclear antibodies (ANA), alkaline phosphatase (ALP), albumin, and/or prothrombin.

The present methods can comprise conducting any suitable metabolic panel test. For example, the present methods can comprise conducting a metabolic panel test that assesses acid/base balance, glucose, sodium, potassium, BUN, creatinine, and/or blood gases.

The present methods can comprise conducting any suitable lipid test. For example, the present methods can comprise conducting a lipid test that assesses ApoA1 and/or ApoB.

The present methods can comprise conducting any suitable test for blood disease. For example, the present methods can comprise assessing a complete blood count that comprises hematocrit, hemoglobin, MCV, lymphocyte count, WBC count, platelets, and reticulocytes.

The present methods can comprise conducting any suitable autoimmune test. For example, the present methods can comprise conducting an autoimmune test that assesses ANA and/or RF.

The present methods can comprise conducting any suitable infectious disease test. For example, the present methods can comprise conducting an infectious disease test that assesses a maker for TB, malaria, and/or dengue.

The present methods can use any suitable device or instrument for conducting a thyroid test, a thyroid panel test, a test for a liver marker, a metabolic panel test, a lipid test, a test for blood count, an autoimmune test, and/or an infectious disease test on a sample from a life or health insurance applicant. For example, the device or instrument can be a clinical chemistry analyzer, a hematology analyzer, or a hand-held device or instrument. The device or instrument can conduct a test within any suitable period of time. For example, the device or instrument can conduct a test within about 1 hour, 2 hours, 4 hours, 8 hours, 16 hours, or 24 hours.

The present methods can be conducted at any suitable location. For example, the thyroid test, the thyroid panel test, the test for a liver marker or disease, the metabolic panel test, the lipid test, the test for complete blood count, the autoimmune test, and/or the infectious disease test can be conducted at a retail clinic, an urgent care clinic, a retail health clinic or a retail pharmacy such as Walgreens.

In some embodiments, further testing can be undertaken at the retail clinic such as Walgreens or urgent care clinic, and can be accomplished in under one-hour time. Such exemplary testing can include a thyroid test) TSH) or thyroid panels (TSH, T4, T3), additional liver testing (GGT, ANA, ALP, albumin, prothrombin), metabolic panels (acid/base balance, glucose, sodium, potassium, BUN, creatinine, blood gases), additional lipid tests (ApoA1, ApoB), and complete blood count (Hematocrit, hemoglobin, MCV, Lymphocyte count, WBC count, platelets, reticulocytes) and autoimmune testing (ANA, RF). Additional testing can be performed for specific populations who may be or have been exposed to other diseases such as TB, malaria, dengue. All of these tests can be performed for convenience and speed within about an hour at a retail health clinic such as an urgent care center or retail pharmacy for purposes of rapidly issuing a life insurance policy. Certain exemplary instruments and tests are currently CLIA waived and can run just a drop of blood. Examples of such instruments include the ABX Pentra 60C and Sysmex POCH 100i for hematology and CBC. The IStat Chem 8 takes a metabolic panel measurement, the Cholestech LDx measure different lipids, liver enzymes and glucose, and the Piccolo POC Blood Chemistry Analyzer measure kidney, liver and metabolic indices.

In some embodiments, full panels of clinical chemistry and hematology tests can now be conducted in a retail pharmacy, urgent care clinic, retail clinics or retail health clinics without the need to send samples to central lab for testing. For example, hand-held clinical chemistry analyzer and hematology analyzer can be used for such tests. This new process eliminates time and assures more rapid underwriting for larger coverage policies. This new process also eliminates the need for testing and sampling at the applicant's home as all testing and sampling would be done at the retail pharmacy or urgent care clinic where the point of presence chemistry and hematology analyzers will be located. All lab information can be collated onto one instrument and/or computer and sent as a complete report to the underwriter from the retail or urgent care setting.

The present methods can be used for assessing any suitable life insurance applications. Life insurance can include all forms of individual and group insurance coverage that contain life contingencies such as term life, whole life, endowment life, universal life, variable life, variable universal life, accidental death, life settlement, pension, and annuities. These contracts can provide for the payment of a beneficiary in the event of a person's death (e.g., term life insurance) or the payment of a beneficiary during the lifetime of an annuitant (e.g., an annuity or pension plan). Exemplary life insurance can include mortgage life insurance, permanent life insurance, term life insurance, universal life insurance (often shortened to UL), variable universal life insurance (often shortened to VUL) and whole life insurance, or whole of life assurance.

The present methods can be used for assessing any suitable health insurance applications. Health insurance can include all forms of individual and group health insurance coverage that involve individual health contingencies such as major medical, HMO, long term care, disability income, limited benefit forms of coverage such as dental care, and accident coverage. These plans can provide either for the reimbursement of an insured for covered health care services (e.g., major medical plans) or the payment of a stated amount of money on a periodic basis in connection with a health contingency (e.g., disability income or long term care insurance). Exemplary health insurance can include accidental death and dismemberment (also known as AD&D), medical insurance, dental insurance, disability insurance (often called DI or disability income insurance), total permanent disability (TPD), and income protection insurance (IPI).

### E. Exemplary Embodiments

In some embodiments, the present invention provides systems and methods for assessing the health of a person, including the mortality and/or morbidity risk of the person.

In some embodiments, the present invention provides for a multiplex, Point-of-Decision, prognostic chip that is comprised of multiple in vitro prognostic tests which, when taken together, are indicators for mortality and morbidity risk that result from the leading causes of debilitating diseases and death such as diabetes, cancer, cardiovascular disease, infectious disease, and drugs of abuse, and can be used to underwrite life or health insurance policies.

Other systems, methods, features, and advantages of the embodiments will be, or will become, apparent to one of ordinary skill in the art upon examination of this disclosure. It is intended that all such additional systems, methods, features, and advantages be included within this description and this summary, be within the scope of the embodiments, and be protected by the claims.

In some embodiments, the present invention provides a vastly improved process and product for assessing the mortality and/or morbidity risk of a prospective client by performing very specific prognostic tests, at the point of application, using a multiplex-chip, populated with optimized tests that can be conducted with a comparatively nominal sample of body fluid, e.g., blood via a finger stick, and which does not require a paramedic to conduct the blood draw, nor client fasting; producing definitive results within 2 hours, 1 hour, 30 minutes or so.

In some embodiments, blood tests for underwriting insurance are generally a subset of standard clinical chemistry panels. This is because up until now, there was no Point-of-Decision testing technology available where very specific tests could be run in a multiplex format resulting in the client and the insurance company obtaining results within minutes of first applying for insurance. Leveraging customized, multiplexing in vitro prognostic chips, specific and prognostic tests can be conducted with significant results while the client is waiting. Additionally, the sample may be taken from whole blood, plasma, urine, serum, or saliva with or without fasting. Additionally, the chip results can be used to electronically establish ratios, such as ApoA1:ApoB and/or can be electronically linked with electronic scales, blood pressure devices, and other underwriting data inputs, either wired or wirelessly.

In some embodiments, in addition to two or more prognostic tests conducted using a blood sample, one or more prognostic tests (e.g., two prognostic tests such as HIV and HCV tests) may be conducted using a separate saliva sample. In this manner, for example, if the multiplex testing requires more sample volume to reach the necessary sensitivities, some of the tests could be run from a separate type of sample that does not require a second finger stick.

Some embodiments may provide systems and methods using a biochip that accepts both blood and also another body fluid. For example, some embodiments may provide a combination saliva and blood biochip, in which the sample collection area of the biochip is partitioned to accept saliva in one section and blood in the other.

The disclosure of embodiments herein has been presented for purposes of illustration and description. It is not intended to be exhaustive or to limit other embodiments to the precise forms disclosed. Many variations and modifications of the embodiments described herein will be apparent to one of ordinary skill in the art in light of the above disclosure. The scope of the embodiments is to be defined only by the claims, and by their equivalents.

In some embodiments, the present invention provides for a multiplex chip that is more prognostic for assessing mortality and morbidity risk than conventional clinical chemistry and hematology panels and can give results within an hour while the insurance client waits.

In some embodiments, the multiplex chip can comprise two or more of the following prognostic tests: HIV, HCV, liver enzymes AST/ALT, nt-probnp, cotinine and/or nicotine, cocaine and/or benzoylecgonine, PSA, ApoA1, ApoB, Hemoglobin A1c, and hsCRP.

In some embodiments, the results can be electronically linked to an electronic blood pressure and scale (e.g., body mass index) measurement for a single collated prognostic report.

In some embodiments, the tests do not require food fasting.

In some embodiments, the chip utilizes blood, plasma, serum, urine, and/or saliva as the sample fluid.

In some embodiments, the chip is configured to accept two or more different body fluids as required for each different assay, where each body fluid is separated from the other and is only used for certain assays on the chip.

In some embodiments, the test results can be simultaneously expressed with a client identifier such as an iris scan, fingerprint, haplotype, or DNA sequence.

In some embodiments, the multiplex chip can comprise two or more first prognostic tests that are conducted using a blood sample, and wherein one or more second prognostic tests are conducted using a separate saliva sample.

In some embodiments, the multiplex chip can comprise one or more second prognostic tests that are an HIV test and an HCV test.

In some embodiments, the multiplex chip can accept blood and another body fluid.

In some embodiments, the multiplex chip can separately accept blood for running certain assays such as NT-proBNP, ApoA1 and ApoB and A1c and separately accepts saliva for running certain assays such as HIV, HCV, cotinine and/or nicotine, cocaine and/or benzoylecgonine.

In some embodiments, the present invention provides a method for collecting more than one body fluid that is loaded onto the chip of the above embodiments.

In some embodiments, the present invention provides a method for collecting more than one finger stick of blood to increase the volume of blood sample to be able to run multiple assays on a chip simultaneously.

In some embodiments, the present invention provides for a finger stick device that simultaneously makes two or more punctures at once to increase the amount of blood sample volume needed to perform tests on a multiplex chip.

The present invention is further illustrated by the following exemplary embodiments:

1. A kit for assessing a life or health insurance applicant, which kit comprises at least two rapid test devices (e.g., singleplex or multiplex rapid test devices), said rapid test devices are configured to assess at least two analytes in a sample derived from a life or health insurance applicant, said at least two analytes being selected from the group consisting of a human immunodeficiency virus (HIV) antigen (e.g., a HIV polypeptide), a HIV polynucleotide, an anti-HIV antibody, a hepatitis C virus (HCV) antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), prostate-specific antigen (PSA), apolipoprotein A-1 (ApoA1), apolipoprotein B (ApoB), Hemoglobin A1c and high-sensitivity C-reactive protein (hsCRP), and preferably, said kit can be used for assessing a life or health insurance applicant at point of presence.

2. The kit of embodiment 1, which comprises at least 3, 4, or 5 rapid test devices, and wherein the rapid test devices are configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP.

3. The kit of embodiment 1, which comprises at least 5 rapid test devices, and wherein the rapid test devices are configured to assess 5 analytes selected from the group consisting of an anti-HIV antibody, an anti-HCV antibody, cotinine or nicotine, cocaine or benzoylecgonine, and Hemoglobin A1c.

4. The kit of any of the embodiments 1-3, wherein at least one of the rapid test devices is selected from the group consisting of a lateral flow test device, a flow through test device, a microfluidic test device, an immunochromatography test device, a single use cartridge or test device, a disposable test device, a self-contained test device, a point of care test device, a microsphere based test device, a nanoparticle based test device, a test strip device, a spot test device, a centrifugal device, and a pump based test device.

5. The kit of any of the embodiments 1-4, wherein at least one of the rapid test devices can be used to conducted a test within about 12 hours.

6. The kit of any of the embodiments 1-5, which comprises at least two lateral flow test devices.

7. The kit of embodiment 6, which comprises at least 3, 4, or 5 lateral flow test devices, and wherein the lateral flow test devices are configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP.

8. The kit of embodiment 6, which comprises at least 5 lateral flow test devices, and wherein the lateral flow test devices are configured to assess 5 analytes selected from the group consisting of an anti-HIV antibody, an anti-HCV antibody, cotinine or nicotine, cocaine or benzoylecgonine, and Hemoglobin A1c.

9. The kit of any of the embodiments 6-8, wherein at least one of the lateral flow test devices comprises a porous matrix that comprises a test zone on the porous matrix, the test zone comprising a test reagent that binds to an analyte or another binding reagent that binds to the analyte, or is an analyte or an analyte analog that competes with an analyte in a sample for binding to a binding reagent for the analyte,

a liquid sample flows laterally along the test device and passes the test zone to form a detectable signal to indicate presence, absence and/or amount of the analyte in the liquid sample, and

the formation of the detectable signal requires the use of a detectable label.

10. The kit of any of the embodiments 6-9, which is to be used in a sandwich assay for the analyte and wherein the test reagent at the test zone binds to the analyte, and a second binding reagent that comprises a detectable label and binds to the analyte and is used,

11. The kit of any of the embodiments 6-9, which is to be used in a sandwich assay for the analyte and wherein the test reagent at the test zone binds to the analyte, and a second binding reagent that comprises a detectable label and binds to another binding reagent that binds to an analyte is used.

12. The kit of any of the embodiments 6-9, which is to be used in a competitive assay for the analyte and wherein the test reagent at the test zone is an analyte or an analyte analog, a second binding reagent that comprises a detectable label and binds to the analyte is used, and the analyte or an analyte analog at the test zone competes with an analyte in the sample for binding to the second binding reagent.

13. The kit of any of the embodiments 6-9, which is to be used in a competitive assay for the analyte and wherein the test reagent at the test zone is an analyte or an analyte analog, a second binding reagent that comprises a detectable label and binds to another binding reagent that binds to an analyte is used, and the analyte or an analyte analog at the test zone competes with an analyte in the sample for binding to the binding reagent that is bound to the second binding reagent.

14. The kit of any of the embodiments 1-13, wherein the analyte is a polypeptide, a polynucleotide or a small molecule, and the test reagent and/or binding reagent that binds to the analyte is an antibody that binds to the polypeptide or small molecule, or another polynucleotide that is substantially complementary to the analyte polynucleotide.

15. The kit of any of the embodiments 6-14, wherein the matrix comprises nitrocellulose, glass fiber, polypropylene, polyethylene (preferably of very high molecular weight), polyvinylidene fluoride, ethylene vinyl acetate, acrylonitrile and/or polytetrafluoro-ethylene.

16. The kit of any of the embodiments 6-15, wherein the matrix is in the form a strip or a circle.

17. The kit of any of the embodiments 6-16, wherein the matrix is a single element or comprises multiple elements.

18. The kit of any of the embodiments 6-17, which further comprises a sample application element upstream from and in fluid communication with the matrix.

19. The kit of any of the embodiments 6-18, which further comprises a liquid absorption element downstream from and in fluid communication with the matrix.

20. The kit of any of the embodiments 6-19, which further comprises a control zone comprising means for indicating proper flow of the liquid sample and/or a valid test result.

21. The kit of any of the embodiments 6-20, wherein at least a portion of the matrix is supported by a solid backing.

22. The kit of any of the embodiments 6-21, wherein a substance is dried on a portion of the matrix upstream from the test zone, the dried substance being capable of being moved by a liquid sample and/or a further liquid to the test zone and/or a control zone to generate a detectable signal, the dried substance being at least one of the second binding reagent that binds to the analyte, the second binding reagent that binds to another binding reagent that binds to the analyte, the analyte or the analyte analog, each of the second binding reagent, analyte or analyte analog comprises a detectable label.

23. The kit of any of the embodiments 6-22, wherein the substance is dried on a conjugate element that is upstream from the test zone.

24. The kit of any of the embodiments 6-23, wherein the substance is located downstream from a sample application place on the test device.

25. The kit of any of the embodiments 6-24, wherein the substance is located upstream from a sample application place on the test device.

26. The kit of any of the embodiments 6-25, wherein the detectable label is a soluble label.

27. The kit of embodiment 26, wherein the soluble label is a soluble enzyme or fluorescent label.

28. The kit of any of the embodiments 6-26, wherein the detectable label is a particle label.

29. The kit of embodiment 28, wherein the particle label is a visible or a non-visible particle label.

30. The kit of embodiment 29, wherein the visible particle label is selected from the group consisting of a colloidal gold label, a latex particle label, a nanoparticle label and a quantum dot label.

31. The kit of embodiment 29, wherein the non-visible particle label is a fluorescent particle.

32. The kit of any of the embodiments 6-31, wherein the substance is dried in the presence of a material that: a) stabilizes the dried substance; b) facilitates solubilization or re-suspension of the dried substance in a liquid; and/or c) facilitates mobility of the dried substance.

33. The kit of embodiment 32, wherein the material is selected from the group consisting of a protein, a peptide, a polynucleotide, a polysaccharide, a sugar, a polymer, a gelatin and a detergent.

34. The kit of any of the embodiments 6-33, wherein a sample liquid alone is used to transport the analyte and/or the substance to the test zone.

35. The kit of any of the embodiments 6-34, wherein a developing liquid is used to transport the analyte and/or the substance to the test zone.

36. The kit of any of the embodiments 6-35, which further comprises a housing that covers at least a portion of the test device, wherein the housing comprises a sample application port to allow sample application upstream from or to the test zone and an optic opening around the test zone to allow signal detection at the test zone.

37. The kit of embodiment 36, wherein the housing covers the entire test device.

38. The kit of embodiment 36, wherein at least a portion of the sample receiving portion of the matrix or the sample application element is not covered by the housing and a sample is applied to the portion of the sample receiving portion of the matrix or the sample application element outside the housing and then transported to the test zone.

39. The kit of any of the embodiments 6-38, wherein at least one of the lateral flow test devices is configured to receive one type of sample, and another lateral flow test device is configured to receive a different type of sample.

40. The kit of embodiment 39, wherein at least one of the lateral flow test devices is configured to receive a blood sample and another lateral flow test device is configured to receive a saliva sample.

41. The kit of any of the embodiments 6-40, wherein at least one of the lateral flow test devices is configured to assess an analyte qualitatively.

42. The kit of any of the embodiments 6-41, wherein at least one of the lateral flow test devices is configured to assess an analyte quantitatively or semi-quantitatively.

43. The kit of any of the embodiments 6-42, wherein at least one of the lateral flow test devices is configured to assess at least two analytes.

44. A system for assessing a life or health insurance applicant, which system comprises the kit of any of the embodiments 1-43.

45. The system of embodiment 44, which further comprises an instruction for using the system for assessing mortality and/or morbidity risk of the life or health insurance applicant, and/or making a decision on the insurance application from the life insurance applicant.

46. The system of embodiment 44 or 45, which further comprises a means for assessing an additional health indicator of the life or health insurance applicant.

47. The system of embodiment 46, wherein the means is used to assess a blood pressure, a body mass index and/or alcohol consumption or alcoholism of the life or health insurance applicant.

48. The system of any of the embodiments 44-47, which further comprises a means for assessing an identifier of the life or health insurance applicant (e.g., an iris scan, a fingerprinting device, a haplotyping device, a nucleic acid, e.g., DNA, sequencer, or a means for assessing voice identification, photograph, electronic signature, or photo identification).

49. The system of any of the embodiments 44-48, which further comprises means for obtaining at least two different types of samples from the life or health insurance applicant.

50. The system of any of the embodiments 44-49, which further comprises means for collecting more than one finger stick of blood from the life or health insurance applicant to increase the volume of blood sample to be able to run multiple assays on the blood sample.

51. The system of any of the embodiments 44-49, which further comprises a finger stick device that simultaneously makes at least two punctures at once to increase the amount of blood sample volume needed to perform multiple assays on the blood sample.

52. The system of any of the embodiments 44-51, which further comprises machine-readable information, e.g., a bar code.

53. The system of embodiment 52, wherein the machine-readable information is comprised in a storage medium, e.g., a RFID device.

54. The system of any of the embodiments 44-53, which further comprises:

a) a reader to assess the detectable signal;

b) a means for recording, storing and/or sending test results in an electronic format;

c) a software program and/or algorithm that collates and/or optimizes all data inputs such as assay results, BMI, application, release form, attending physician statement (APS), medical information bureau (MIB) record, motor vehicle record (MVR), credit report and prescription or transaction history record into one electronically transferrable or printable file; and/or

d) an apparatus, software and/or algorism for forensic voice analysis that detects emotions in the human voice.

55. The system of embodiment 54, wherein the reader is a fluorescent reader, a colorimetric reader, or luminescent reader.

56. The system of embodiment 55, wherein the fluorescent reader is a laser based or a light emitting diode (LED) based fluorescent reader.

57. A lateral flow test device for assessing a life or health insurance applicant, which device is configured to assess at least two analytes in a sample derived from a life or health insurance applicant, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and

said device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to assess said at least two analytes in a sample, and the formation of said detectable signal requires the use of a detectable label.

58. The device of embodiment 57, which is configured to assess 3, 4, or 5 analytes selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP.

59. The device of embodiment 58, which is configured to assess 5 analytes selected from the group consisting of an anti-HIV antibody, an anti-HCV antibody, nicotine or cocaine, cocaine or benzoylecgonine, and Hemoglobin A1c.

60. The device of any of the embodiments 57-59, which is to be used in a sandwich assay for the analytes and wherein the test reagents at the test locations bind to the analytes, and a binding reagent that comprises a detectable label and binds to the analytes is used.

61. The device of any of the embodiments 57-59, which is to be used in a sandwich assay for the analytes and wherein test reagents at the test locations bind to the analyte, and a binding reagent that comprises a detectable label and binds to another binding reagent that binds to the analyte is used.

62. The device of embodiment 60 or 61, wherein a single binding reagent that comprises a detectable label and binds to the analytes is used.

63. The device of embodiment 60 or 61, wherein multiple binding reagents that comprise a detectable label and bind to the analytes are used.

64. The device of any of the embodiments 57-59, which is to be used in a competitive assay for the analytes and wherein the test reagents at the test locations are analytes or an analyte analogs, a second binding reagent that comprises a detectable label and binds to the analytes is used, and the analytes or an analyte analogs at the test locations compete with analytes in the sample for binding to the second binding reagent.

65. The device of any of the embodiments 57-59, which is to be used in a competitive assay for the analytes and wherein the test reagents at the test locations are analytes or an analyte analogs, a second binding reagent that comprises a detectable label and binds to another binding reagent that binds to the analytes is used, and the analytes or an analyte analogs at the test locations compete with the analytes in the sample for binding to the binding reagent that is bound to the second binding reagent.

66. The device of embodiment 64 or 65, wherein a single second binding reagent is used.

67. The device of embodiment 64 or 65, wherein multiple second binding reagents are used.

68. The device of any of the embodiments 57-67, wherein the analytes are polypeptides, polynucleotides or small molecules, and the test reagents and/or binding reagents that bind to the analytes are antibodies that bind to the polypeptides or small molecules, or another polynucleotide that is substantially complementary to the analyte polynucleotides.

69. The device of any of the embodiments 57-68, wherein the matrix comprises nitrocellulose, glass fiber, polypropylene, polyethylene (preferably of very high molecular weight), polyvinylidene fluoride, ethylene vinyl acetate, acrylonitrile and/or polytetrafluoro-ethylene.

70. The device of any of the embodiments 57-69, wherein the matrix is in the form a strip or a circle.

71. The device of any of the embodiments 57-70, wherein the matrix is a single element or comprises multiple elements.

72. The device of any of the embodiments 57-71, which further comprises a sample application element upstream from and in fluid communication with the matrix.

73. The device of any of the embodiments 57-72, which further comprises a liquid absorption element downstream from and in fluid communication with the matrix.

74. The device of any of the embodiments 57-73, which further comprises a control zone comprising means for indicating proper flow of the liquid sample and/or a valid test result.

75. The device of any of the embodiments 57-74, wherein at least a portion of the matrix is supported by a solid backing.

76. The device of any of the embodiments 57-75, wherein a substance is dried on a portion of the matrix upstream from the test locations, the dried substance being capable of being moved by a liquid sample and/or a further liquid to the test locations and/or a control location to generate a detectable signal, the dried substance being at least one of the second binding reagent that binds to the analyte, the second binding reagent that binds to another binding reagent that binds to the analyte, the analyte or the analyte analog, each of the second binding reagent, analyte or analyte analog comprises a detectable label.

77. The device of embodiment 76, wherein a single substance is dried on a portion of the matrix upstream from the test locations.

78. The device of embodiment 76, wherein multiple substances are dried on a portion of the matrix upstream from the test locations.

79. The device of any of the embodiments 76-78, wherein the substance is dried on a conjugate element that is upstream from the test zone.

80. The device of any of the embodiments 76-79, wherein the substance is located downstream from a sample application place on the test device.

81. The device of any of the embodiments 76-79, wherein the substance is located upstream from a sample application place on the test device.

82. The device of any of the embodiments 57-81, wherein the detectable label is a soluble label.

83. The device of embodiment 82, wherein the soluble label is a soluble enzyme or fluorescent label.

84. The device of any of the embodiments 57-81, wherein the detectable label is a particle label.

85. The device of embodiment 84, wherein the particle label is a visible or a non-visible particle label.

86. The device of embodiment 85, wherein the visible particle label is selected from the group consisting of a colloidal gold label, a latex particle label, a nanoparticle label and a quantum dot label.

87. The device of embodiment 85, wherein the non-visible particle label is a fluorescent particle.

88. The device of any of the embodiments 76-87, wherein the substance is dried in the presence of a material that: a) stabilizes the dried substance; b) facilitates solubilization or re-suspension of the dried substance in a liquid; and/or c) facilitates mobility of the dried substance.

89. The device of embodiment 88, wherein the material is selected from the group consisting of a protein, a peptide, a polynucleotide, a polysaccharide, a sugar, a polymer, a gelatin and a detergent.

90. The device of any of the embodiments 57-89, wherein a sample liquid alone is used to transport the analytes and/or the substance to the test locations.

91. The device of any of the embodiments 57-90, wherein a developing liquid is used to transport the analytes and/or the substance to the test locations.

92. The device of any of the embodiments 57-91, which further comprises a housing that covers at least a portion of the test device, wherein the housing comprises a sample application port to allow sample application upstream from or to the test locations and an optic opening around the test locations to allow signal detection at the test locations.

93. The device of embodiment 92, wherein the housing covers the entire test device.

94. The device of embodiment 93, wherein at least a portion of the sample receiving portion of the matrix or the sample application element is not covered by the housing and a sample is applied to the portion of the sample receiving portion of the matrix or the sample application element outside the housing and then transported to the test locations.

95. The device of any of the embodiments 57-94, which is configured to receive at least two different types of samples.

96. The device of embodiment 95, which is configured to receive a blood sample and a saliva sample.

97. The device of any of the embodiments 57-96, wherein the test locations are in the same liquid flow pathway.

98. The device of any of the embodiments 57-96, wherein the test locations are in the different liquid flow pathways.

99. The device of embodiment 98, wherein the different liquid flow pathways are shielded from each other.

100. The device of any of the embodiments 57-99, which is configured to assess at least one of the analytes qualitatively.

101. The device of any of the embodiments 57-99, which is configured to assess at least one of the analytes quantitatively or semi-quantitatively.

102. A system for assessing a life or health insurance applicant, which system comprises the device of any of the embodiments 57-101.

103. The system of embodiment 102, which further comprises an instruction for using the system for assessing mortality and/or morbidity risk of the life insurance or health applicant, and/or making a decision on the insurance application from the life insurance applicant.

104. The system of embodiment 102 or 103, which further comprises a means for assessing an additional health indicator of the life or health insurance applicant.

105. The system of embodiment 104, wherein the means is used to assess a blood pressure, a body mass index and/or alcohol consumption or alcoholism of the life or health insurance applicant.

106. The system of any of the embodiments 102-105, which further comprises a means for assessing an identifier of the life or health insurance applicant.

107. The system of embodiment 106, wherein the means is an iris scan, a fingerprinting device, a haplotyping device, a DNA sequencer, or a means for assessing voice identification, photograph, electronic signature, or photo identification.

108. The system of any of the embodiments 102-108, which further comprises means for obtaining at least two different types of samples from the life or health insurance applicant.

109. The system of any of the embodiments 102-108, which further comprises means for collecting more than one finger stick of blood from the life or health insurance applicant to increase the volume of blood sample to be able to run multiple assays on the blood sample.

110. The system of any of the embodiments 102-108, which further comprises a finger stick device that simultaneously makes at least two punctures at once to increase the amount of blood sample volume needed to perform multiple assays on the blood sample.

111. The system of any of the embodiments 102-110, which further comprises machine-readable information, e.g., a bar code.

112. The system of embodiment 111, wherein the machine-readable information is comprised in a storage medium, e.g., a RFID device.

113. The system of any of the embodiments 102-112, which further comprises a reader to assess the detectable signal, a means for recording, storing and/or sending test results in an electronic format, and/or a software program and/or algorithm that collates and optimizes all data inputs such as assay results, BMI, application, release form, attending physician statement (APS), medical information bureau (MIB) record, motor vehicle record (MVR), credit report and prescription or transaction history record into one electronically transferrable or printable file, and/or an apparatus, software and/or algorism for forensic voice analysis that detects emotions in the human voice.

114. The system of embodiment 113, wherein the reader is a fluorescent reader, a colorimetric reader, or luminescent reader.

115. The system of embodiment 114, wherein the fluorescent reader is a laser based or a light emitting diode (LED) based fluorescent reader.

116. A method for assessing a life or health insurance applicant, which method comprises:

a) assessing the presence, absence and/or amount of at least two analytes in a sample derived from a life or health insurance applicant using a rapid test, said at least two analytes being selected from the group consisting of a HIV antigen (e.g., a HW polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and

b) assessing an insurance application from said life or health insurance applicant based on the test results obtained in step a).

117. The method of embodiment 116, which is conducted using the kit of any of the embodiments 1-43, the system of any of the embodiments 44-56, the device of any of the embodiments 57-101, or the system of any of the embodiments 102-115.

118. The method of embodiment 117, which is conducted using the kit of any of the embodiments 6-43 or the system of any of the embodiments 39-51, and comprises:

a) contacting a liquid sample with at least one of the lateral flow test devices in the kit of any of the embodiments 6-43 or the system of any of the embodiments 44-56, wherein the liquid sample is applied to a site of the test device upstream of the test zone;

b) transporting an analyte, if present in the liquid sample, and a labeled reagent to the test zone;

c) assessing a detectable signal at the test zone to determine the presence, absence and/or amount of the analyte; and

wherein steps a)-c) are conducted using at least two of the lateral flow test devices in the kit of any of the embodiments 6-43 or the system of any of the embodiments 44-56 to assessing the presence, absence and/or amount of at least two analytes in the sample.

119. The method of embodiment 117, which is conducted using the device of any of the embodiments 57-101 or the system of any of the embodiments 102-115, and comprises:

a) contacting a liquid sample with the device of any of the embodiments 57-101 or the system of any of the embodiments 102-115, wherein the liquid sample is applied to a site of the test device upstream of the test locations;

b) transporting multiple analytes, if present in the liquid sample, and a labeled reagent to the test locations; and

c) assessing a detectable signal at the test locations to determine the presence, absence and/or amount of the analyte in the sample.

120. The method of embodiment 118 or 119, wherein the liquid sample and the labeled reagent are premixed to form a mixture and the mixture is applied to the test device.

121. The method of embodiment 120, which further comprises a washing step after the mixture is applied to the test device.

122. The method of embodiment 121, wherein the test device comprises a liquid container comprising a washing liquid and the washing step comprises releasing the washing liquid from the liquid container.

123. The method of any of the embodiments 118-122, wherein the test device comprises a dried labeled reagent before use and the dried labeled reagent is solubilized or re-suspended, and transported to the test zone or locations by the liquid sample.

124. The method of embodiment 123, wherein the dried labeled reagent is located downstream from the sample application site, and the dried labeled reagent is solubilized or re-suspended, and transported to the test zone or location by the liquid sample.

125. The method of embodiment 123, wherein the dried labeled reagent is located upstream from the sample application site, and the dried labeled reagent is solubilized or re-suspended, and transported to the test zone or locations by another liquid.

126. The method of any of the embodiments 123-125, wherein the labeled reagent is solubilized or re-suspended, and transported to the test zone or location by the liquid sample alone.

127. The method of any of the embodiments 123-125, wherein the analyte(s) and/or labeled reagent are solubilized or re-suspended, and transported to the test zone or location by another liquid.

128. The method of any of the embodiments 116-127, wherein the liquid sample is a body fluid sample.

129. The method of embodiment 128, wherein the body fluid sample is selected from the group consisting of a whole blood, a serum, a plasma, a fresh blood, a blood not containing an anti-coagulate, a urine and a saliva sample.

130. The method of embodiment 129, wherein a liver enzyme alanine transaminase (ALT), a liver enzyme aspartate transaminase (AST), nt-probnp (or NT-proBNP), ApoA1, ApoB, Hemoglobin A1c or hsCRP is assessed using a blood sample.

131. The method of embodiment 129, wherein a HIV antigen, an anti-HIV antibody, a HCV antigen, an anti-HCV antibody, cotinine or nicotine, or cocaine or benzoylecgonine is assessed using a saliva sample.

132. The method of any of the embodiments 116-131, wherein at least 3, 4, or 5 analytes are assessed.

133. The method of any of the embodiments 116-132, which comprises assessing 5 analytes selected from the group consisting of an anti-HIV antibody, an anti-HCV antibody, cotinine or nicotine, cocaine or benzoylecgonine and Hemoglobin A1c.

134. The method of embodiment 133, wherein an anti-HIV antibody, an anti-HCV antibody, and Hemoglobin A1c are assessed using a sandwich assay.

135. The method of embodiment 133, wherein cotinine or nicotine and cocaine or benzoylecgonine are assessed using a competitive assay.

136. The method of any of the embodiments 116-135, which is used to assess mortality and/or morbidity of a life or health insurance applicant.

137. The method of any of the embodiments 116-136, wherein at least one of the analytes is assessed qualitatively.

138. The method of any of the embodiments 116-136, wherein at least one of the analytes is assessed quantitatively or semi-quantitatively.

139. The method of any of the embodiments 116-138, wherein the detectable signal is assessed by a reader.

140. The method of embodiment 139, wherein the detectable signal is a fluorescent signal and the fluorescent signal is assessed by a fluorescent reader.

141. The method of embodiment 140, wherein the fluorescent reader is a laser based or a light emitting diode (LED) based fluorescent reader.

142. The method of any of the embodiments 116-141, wherein a sample derived from a life or health insurance applicant without fasting (e.g., food fasting) is used.

143. The method of any of the embodiments 116-142, which is conducted within an hour.

144. The method of any of the embodiments 116-143, which further comprises assessing an additional health indicator of a life or health insurance applicant.

145. The method of embodiment 144, wherein the additional health indicator is a blood pressure, a body mass index and/or alcohol consumption or alcoholism of the life or health insurance applicant.

146. The method of any of the embodiments 116-145, which further comprises assessing an identifier of a life or health insurance applicant, and/or conducting forensic voice analysis of the voice of an applicant.

147. The method of embodiment 146, wherein the identifier of a life or health insurance applicant is assessed by an iris scanning, fingerprinting, haplotyping, or DNA sequencing, or by assessing voice identification, photograph, electronic signature, or photo identification of the applicant.

148. The method of any of the embodiments 116-147, which is conducted at the point of presence.

149. The method of any of the embodiments 116-148, wherein the test results and collated information are transmitted to an insurance decision maker within about 24 hours from the time when a sample is obtained from the applicant.

150. The kit of any of the embodiments 1-43 or the system of any of the embodiments 44-56, which further comprises a solid medium for collecting, drying and/or storing a sample derived from a life or health insurance applicant wherein said collected, dried and/or stored sample can be used in a test.

151. The kit or system of embodiment 150, wherein the solid medium is a porous solid medium.

152. The kit or system of embodiment 151, wherein the porous solid medium comprises filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene.

153. The kit or system of embodiment 150, wherein the solid medium is a non-porous solid medium.

154. The kit or system of embodiment 153, wherein the non-porous solid medium comprises a plastic material or a glass slide.

155. The kit or system of any of the embodiments 150-154, wherein the solid medium is contained in a container or a device, or is at least a part of a surface of a container or device.

156. The kit or system of embodiment 155, wherein the container is a tube or a microtiter plate.

157. The kit or system of any of the embodiments 150-156, wherein the dried sample is a dried body fluid sample.

158. The kit or system of embodiment 157, wherein the body fluid sample is selected from the group consisting of a whole blood, a serum, a plasma, a fresh blood, a blood not containing an anti-coagulate, a urine and a saliva sample.

159. The kit or system of any of the embodiments 150-158, wherein the sample comprises an analyte that is a polypeptide, a polynucleotide or a small molecule.

160. The kit or system of any of the embodiments 150-159, wherein the sample is collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content.

161. The kit or system of embodiment 160, wherein the material is selected from the group consisting of a protein, a peptide, a polynucleotide, a polysaccharide, a sugar, a polymer, a gelatin and a detergent.

162. The kit or system of any of the embodiments 150-161, wherein the solid medium comprises a collected, dried and/or stored sample.

163. The kit or system of embodiment 162, wherein the sample is dried on a solid medium as a dried spot.

164. The kit or system of embodiment 162 or 163, wherein the collected, dried and/or stored sample is stored for a short term, e.g., about 1, 2, 3, 4, 5, 6, 7, 8, 9 or 10 days.

165. The kit or system of embodiment 162 or 163, wherein the collected, dried and/or stored sample is stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

166. The kit or system of any of the embodiments 162-165, wherein the collected, dried and/or stored sample is retested for an analyte that has been tested before for assessing a life or health insurance applicant from which the sample is derived.

167. The kit or system of any of the embodiments 162-165, wherein the collected, dried and/or stored sample is tested for an analyte that has not been tested before.

168. The kit or system of any of the embodiments 162-165, wherein the collected, dried and/or stored sample is tested for assessing an identifier, e.g., a personal identifier, of the life or health insurance applicant.

169. The kit or system of any of the embodiments 150-168, which further comprises a means for collecting additional sample from a life or health insurance applicant to be stored on a solid medium.

170. The device of any of the embodiments 57-101 or the system of any of the embodiments 102-115, which further comprises a solid medium for collecting, drying and/or storing a sample derived from a life or health insurance applicant wherein said collected, dried and/or stored sample can be used in a test.

171. The device or system of embodiment 170, wherein the solid medium is a porous solid medium.

172. The device or system of embodiment 171, wherein the porous solid medium comprises filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene.

173. The device or system of embodiment 170, wherein the solid medium is a non-porous solid medium.

174. The device or system of embodiment 173, wherein the non-porous solid medium comprises a plastic material or a glass slide.

175. The device or system of any of the embodiments 170-174, wherein the solid medium is contained in a container or a device, or is at least a part of a surface of a container or device.

176. The device or system of embodiment 175, wherein the container is a tube or a microtiter plate.

177. The device or system of any of the embodiments 170-176, wherein the dried sample is a dried body fluid sample.

178. The device or system of embodiment 177, wherein the body fluid sample is selected from the group consisting of a whole blood, a serum, a plasma, a fresh blood, a blood not containing an anti-coagulate, a urine and a saliva sample.

179. The device or system of any of the embodiments 170-178, wherein the sample comprises an analyte that is a polypeptide, a polynucleotide or a small molecule.

180. The device or system of any of the embodiments 170-179, wherein the sample is collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content.

181. The device or system of embodiment 180, wherein the material is selected from the group consisting of a protein, a peptide, a polynucleotide, a polysaccharide, a sugar, a polymer, a gelatin and a detergent.

182. The device or system of any of the embodiments 170-181, wherein the solid medium comprises a dried sample.

183. The device or system of embodiment 182, wherein the sample is dried on a solid medium as a dried spot.

184. The device or system of embodiment 182 or 183, wherein the collected, dried and/or stored sample is stored for a short term, e.g., about 1, 2, 3, 4, 5, 6, 7, 8, 9 or 10 days.

185. The device or system of embodiment 182 or 183, wherein the collected, dried and/or stored sample is stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

186. The device or system of any of the embodiments 182-185, wherein the collected, dried and/or stored sample is retested for an analyte that has been tested before for assessing a life or health insurance applicant from which the sample is derived.

187. The device or system of any of the embodiments 182-185, wherein the collected, dried and/or stored sample is tested for an analyte that has not been tested before.

188. The device or system of any of the embodiments 182-185, wherein the collected, dried and/or stored sample is tested for assessing an identifier of the life or health insurance applicant.

189. The device or system of any of the embodiments 170-188, which further comprises a means for collecting additional sample from a life or health insurance applicant to be stored on a solid medium.

190. The method of any of the embodiments 116-149, which further comprise collecting, drying and/or storing a sample derived from a life or health insurance applicant on or in a solid medium wherein said collected, dried and/or stored sample can be used in a test.

191. The method of embodiment 190, wherein the solid medium is a porous solid medium.

192. The method of embodiment 191, wherein the porous solid medium comprises filter paper, nitrocellulose, glass fiber, polypropylene, polyethylene (preferably of very high molecular weight), polyvinylidene fluoride, ethylene vinyl acetate, acrylonitrile and/or polytetrafluoro-ethylene.

193. The method of embodiment 190, wherein the solid medium is a non-porous solid medium.

194. The method of embodiment 193, wherein the non-porous solid medium comprises a plastic material or a glass slide.

195. The method of any of the embodiments 190-194, wherein the solid medium is contained in a container or a device, or is at least a part of a surface of a container or device.

196. The method of embodiment 195, wherein the container is a tube or a microtiter plate.

197. The method of any of the embodiments 190-196, wherein the dried sample is a dried body fluid sample.

198. The method embodiment 197, wherein the body fluid sample is selected from the group consisting of a whole blood, a serum, a plasma, a fresh blood, a blood not containing an anti-coagulate, a urine and a saliva sample.

199. The method of any of the embodiments 190-198, wherein the sample comprises an analyte that is a polypeptide, a polynucleotide or a small molecule.

200. The method of any of the embodiments 190-199, wherein the sample is collected, dried and/or stored on or in a solid medium in the presence of a material that: a) stabilizes the collected, dried and/or stored sample or its content, e.g., an analyte; b) facilitates solubilization or re-suspension of the collected, dried and/or stored sample or its content in a liquid; and/or c) facilitates mobility of the collected, dried and/or stored sample or its content.

201. The method of embodiment 200, wherein the material is selected from the group consisting of a protein, a peptide, a polynucleotide, a polysaccharide, a sugar, a polymer, a gelatin and a detergent.

202. The method of any of the embodiments 190-201, wherein the sample is dried on a solid medium as a dried spot.

203. The method of any of the embodiments 190-202, wherein the collected, dried and/or stored sample is stored for a short term, e.g., about 1, 2, 3, 4, 5, 6, 7, 8, 9 or 10 days.

204. The method of any of the embodiments 190-202, wherein the collected, dried and/or stored sample is stored for a long term, e.g., about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

205. The method of any of the embodiments 190-204, wherein the collected, dried and/or stored sample is retested for an analyte that has been tested before for assessing a life or health insurance applicant from which the sample is derived.

206. The method of any of the embodiments 190-204, wherein the collected, dried and/or stored sample is tested for an analyte that has not been tested before.

207. The method of any of the embodiments 190-204, wherein the collected, dried and/or stored sample is tested for assessing an identifier of the life or health insurance applicant.

208. The method of any of the embodiments 190-207, which further comprises collecting additional sample from a life or health insurance applicant to be stored on a solid medium.

209. The system of any of the embodiments 44-56 and 102-115, which further comprises a device or an instrument for conducting a thyroid test, a thyroid panel test, a test for a liver marker or disease, a metabolic panel test, a lipid test, a test for blood count, an autoimmune test, and/or an infectious disease test on a sample from a life or health insurance applicant.

210. The system of embodiment 209, wherein the thyroid test assesses thyroid function using thyroid-stimulating hormone (TSH).

211. The system of embodiment 209, wherein the thyroid panel test assesses TSH, T4, and/or T3.

212. The system of embodiment 209, wherein the test for a liver disease assesses GGT, ANA, ALP, albumin, and/or prothrombin.

213. The system of embodiment 209, wherein the metabolic panel test assesses acid/base balance, glucose, sodium, potassium, BUN, creatinine, and/or blood gases.

214. The system of embodiment 209, wherein the lipid test assesses ApoA1 and/or ApoB.

215. The system of embodiment 209, wherein the test for blood count assesses complete blood count that comprises Hematocrit, hemoglobin, MCV, Lymphocyte count, WBC count, platelets, and reticulocytes.

216. The system of embodiment 209, wherein the autoimmune test assesses ANA and/or RF.

217. The system of embodiment 209, wherein the infectious disease test assesses a maker for TB, malaria, and/or dengue.

218. The system of any of the embodiments 209-217, wherein the device or instrument is a clinical chemistry analyzer, a hematology analyzer, or a hand-held device or instrument.

219. The system of any of the embodiments 209-218, wherein the device or instrument can conduct a test within about one hour.

220. The system of any of the embodiments 102-115 and 170-189, which further comprises a device or an instrument for conducting a thyroid test, a thyroid panel test, a test for a liver marker or disease, a metabolic panel test, a lipid test, a test for blood count, an autoimmune test, and/or an infectious disease test on a sample from a life or health insurance applicant.

221. The system of embodiment 220, wherein the thyroid test assesses thyroid function using thyroid-stimulating hormone (TSH).

222. The system of embodiment 220, wherein the thyroid panel test assesses TSH, T4, and/or T3.

223. The system of embodiment 220, wherein the test for a liver disease assesses GGT, ANA, ALP, albumin, and/or prothrombin.

224. The system of embodiment 220, wherein the metabolic panel test assesses acid/base balance, glucose, sodium, potassium, BUN, creatinine, and/or blood gases.

225. The system of embodiment 220, wherein the lipid test assesses ApoA1 and/or ApoB.

226. The system of embodiment 220, wherein the test for blood count assesses complete blood count that comprises Hematocrit, hemoglobin, MCV, Lymphocyte count, WBC count, platelets, and reticulocytes.

227. The system of embodiment 220, wherein the autoimmune test assesses ANA and/or RF.

228. The system of embodiment 220, wherein the infectious disease test assesses a maker for TB, malaria, and/or dengue.

229. The system of any of the embodiments 220-228, wherein the device or instrument is a clinical chemistry analyzer, a hematology analyzer, or a hand-held device or instrument.

230. The system of any of the embodiments 220-229, wherein the device or instrument can conduct a test within about one hour.

231. The method of any of the embodiments 116-149 and 190-208, which further comprises conducting a thyroid test, a thyroid panel test, a test for a liver marker or disease, a metabolic panel test, a lipid test, a test for blood count, an autoimmune test, and/or an infectious disease test on a sample from a life or health insurance applicant.

232. The method of embodiment 231, wherein the thyroid test assesses thyroid function using thyroid-stimulating hormone (TSH).

233. The method of embodiment 231, wherein the thyroid panel test assesses TSH, T4, and/or T3.

234. The method of embodiment 231, wherein the test for a liver disease assesses GGT, ANA, ALP, albumin, and/or prothrombin.

235. The method of embodiment 231, wherein the metabolic panel test assesses acid/base balance, glucose, sodium, potassium, BUN, creatinine, and/or blood gases.

236. The method of embodiment 231, wherein the lipid test assesses ApoA1 and/or ApoB.

237. The method of embodiment 231, wherein the test for blood count assesses complete blood count that comprises Hematocrit, hemoglobin, MCV, Lymphocyte count, WBC count, platelets, and reticulocytes.

238. The method of embodiment 231, wherein the autoimmune test assesses ANA and/or RF.

239. The method of embodiment 231, wherein the infectious disease test assesses a maker for TB, malaria, and/or dengue.

240. The method of any of the embodiments 231-239, wherein the thyroid test, the thyroid panel test, the test for a liver marker or disease, the metabolic panel test, the lipid test, the test for blood count, the autoimmune test, and/or the infectious disease test is conducted using a clinical chemistry analyzer, a hematology analyzer, or a hand-held device or instrument.

241. The method of any of the embodiments 231-240, wherein the thyroid test, the thyroid panel test, the test for a liver marker or disease, the metabolic panel test, the lipid test, the test for blood count, the autoimmune test, and/or the infectious disease test is conducted within about one hour.

242. The method of any of the embodiments 231-241, wherein the thyroid test, the thyroid panel test, the test for a liver marker or disease, the metabolic panel test, the lipid test, the test for blood count, the autoimmune test, and/or the infectious disease test is conducted at a retail clinic, an urgent care clinic, a retail health clinic or a retail pharmacy.

243. The kit or system of embodiment 169, wherein the means comprises a device for collecting a sample to be dried or stabilized for storage purpose.

244. The kit or system of embodiment 243, wherein the dried or stabilized sample is suitable for an optional test that can be completed at a later time, e.g., an optional test for assessing optional insurance coverage.

245. The device or system of embodiment 189, wherein the means comprises a device for collecting a sample to be dried or stabilized for storage purpose.

246. The device or system of embodiment 245, wherein the dried or stabilized sample is suitable for an optional test that can be completed at a later time, e.g., an optional test for assessing optional insurance coverage.

247. The method of embodiment 208, wherein the additional sample is collected using a device and the collected sample is dried or stabilized for storage purpose.

248. The method of embodiment 247, wherein the dried or stabilized sample is further tested, e.g., for assessing optional insurance coverage.

### F. Examples

Five experiment sets are described in the following examples. The work was performed as part of the effort to demonstrate the detection of HCV, HIV, A1C and cocaine in whole blood samples, and cotinine in saliva samples. Existing kits were used to test 10 random whole blood samples and 2 positive samples for HCV, HIV, A1C and Cocaine, and 2 saliva samples for cotinine.

### Example 1

### Detecting HIV, A1c and Cocaine/Benzoylecgonine in Whole Blood Samples

Introduction

Three experiment sets are described in this Example. The work was performed as part of the effort to demonstrate the detection of HIV, A1C and Cocaine in whole blood samples. Existing kits were used to test 10 random whole blood samples and 2 positive samples for each target.

### Materials and Methods

### Materials

The materials used in the experiments are listed below:
Bayer A1CNow+Multitest, CliaWaived, PN: BAYER-3024, LN: 1217054;
ACON 1-step Cocaine Test Cards, CliaWaived, PN: DCO-114, LN: COC2010002; and
Oraquick HIV 1-2 Advance Rapid Test Kit, CliaWaived, PN: MUR-01L7425, LN: 6634367.

### Reagents

The reagents used in the experiments are listed below:
Bezoylecgonine Standard, Sigma, PN: B8900, LN: SLBB2510;
HQ Chex (A1C Positive Control), Fisher, PN: 11-716-305, LN: 30070564; and
Whole Blood Disodium EDTA, Bioreclamation, PN: HMWBEDTA.

TABLE 1

| LOT# | Gender | AGE | RACE |
|---|---|---|---|
| BRH687840 | MALE | 55 | Hispanic |
| BRH687841 | MALE | 53 | Caucasian |
| BRH687842 | MALE | 50 | Black |
| BRH687843 | MALE | 46 | Caucasian |
| BRH687844 | MALE | 50 | Hispanic |
| BRH687845 | MALE | 33 | Black |
| BRH687846 | MALE | 43 | Caucasian |
| BRH687847 | MALE | 59 | Black |
| BRH687848 | MALE | 52 | Black |
| BRH687849 | MALE | 41 | Hispanic |

Human Plasma K2 EDTA HIV, Bioreclamation, PN: HMPLEDTA-HIV

TABLE 2

| LOT# | Gender | AGE | RACE |
|---|---|---|---|
| BRH688577 | MALE | 44 | Black |
| BRH688578 | MALE | 36 | Black |

### Equipment

The equipment used in the experiments is listed below:
Centrifuge, Sorvall, Asset No: CE003, Model No: 75002441, Serial No: 40670922; and
Pipettes, Asset No: P52-55, P9-12, P60-62.

### Experimental Methods

### A1C Detection

All reagents and materials were brought to room temperature. The blood was mixed gently before pipetting 20 uL onto a glass slide. The blood was then drawn up into the blood collector until the inner tube was full and then inserted into the sampler body. The blood was mixed with the sampler buffer by shaking the container and the test cartridge was inserted into the monitor and laid on a flat surface. When the monitor read “SMPL” the base of the sampler body was removed and the sampler body was inserted into the test cartridge. The top of the container was pushed down to release the sample. The results were recorded after 5 minutes.

### Cocaine/Benzoylecgonine Detection

The materials and reagents were brought to room temperature. The blood was centrifuged and the plasma was pipetted into a tube and the Benzoylecgonine was added into two of the plasma samples to a final concentration of 350 ng/ml. The test card cap was removed and the tip was immersed in the plasma for 15-20 seconds. The cap was replaced and the test card was laid flat and results were recorded at 5 minutes.

### HIV Antibody Detection

All materials and reagents were brought to room temperature. The blood/plasma was gently mixed, and the Specimen Collection Loop was dipped into the tube of blood/plasma. The loop was completely filled with blood and then inserted into the vial. The loop was stirred into the Developer Solution and discarded. The flat pad of the device was immersed in the sample and results were recorded after 20 minutes.

### Experimental Results

Table 3 below shows the data collected using the kits for the detection of HIV, Cocaine, and A1C. The bolded results indicate high or positive results.

TABLE 3

| DCN # | LOT# | % A1C | Cocaine | HIV |
|---|---|---|---|---|
| 40 | BRH687840 | 5.4 | Negative | Negative |
| 41 | BRH687841 | 5.8 | Negative | Negative |
| 42 | BRH687842 | 4.9 | Negative | Negative |
| 43 | BRH687843 | 4.6 | Negative | Negative |
| 44 | BRH687844 | 4.9 | Negative | Negative |
| 45 | BRH687845 | 5.5 | Negative | Negative |
| 46 | BRH687846 | 5.6 | Negative | Negative |
| 47 | BRH687847 | 5.4 | Positive | Negative |
| 48 | BRH687848 | 5.4 | Negative | Negative |
| 49 | BRH687849 | 5.2 | Negative | Negative |
| 50 | BRH687840 | 5.7 | N/A | N/A |
| | (20 uL + 1 Drop L3) | | | |
| 51 | L3 | 5.8 | N/A | N/A |
| 52 | BRH687840 | N/A | Positive | N/A |
| | (350 ng/ml COC) | | | |
| 53 | BRH687841 | N/A | Positive | N/A |
| | (350 ng/ml COC) | | | |
| 77 | BRH688577 | N/A | N/A | Positive |
| 78 | BRH688578 | N/A | N/A | Positive |

### Conclusions

The individuals numbered 41, 50, and 51 had high A1C percentages which indicated increased risk for diabetes. The individuals numbered 47, 52, and 53, tested positive for cocaine, and individuals 77 and 78 were HIV positive.

### Example 2

### Rapid Assay for HCV

Introduction

The work was performed as part of the effort to demonstrate the rapid detection of HCV in whole blood samples. The Oraquick HCV Rapid Antibody kit was used to test 10 random and 2 positive whole blood samples for HCV.

### Materials and Methods

### Materials

The materials used in the experiments are listed below:
OraQuick HCV Rapid Antibody Test, CliaWaived, PN: ORA-1001-0181, LN: 6631745.

### Reagents

The reagents used in the experiments are listed below:
Whole Blood Disodium EDTA, Bioreclamation, PN: HMWBEDTA.

TABLE 4

| LOT# | Gender | AGE | RACE |
|---|---|---|---|
| BRH687840 | MALE | 55 | Hispanic |
| BRH687841 | MALE | 53 | Caucasian |
| BRH687842 | MALE | 50 | Black |
| BRH687843 | MALE | 46 | Caucasian |
| BRH687844 | MALE | 50 | Hispanic |
| BRH687845 | MALE | 33 | Black |
| BRH687846 | MALE | 43 | Caucasian |
| BRH687847 | MALE | 59 | Black |
| BRH687848 | MALE | 52 | Black |
| BRH687849 | MALE | 41 | Hispanic |

Human Plasma K2 EDTA HCV+, Bioreclamation, PN: HMPLEDTA2-HCV+

TABLE 5

| LOT# | Gender | AGE | RACE |
|---|---|---|---|
| BRH689636 | MALE | 41 | Caucasian |
| BRH689637 | MALE | 46 | Caucasian |

### Experimental Methods

### HCV Detection

All materials and reagents were brought to room temperature. The blood/plasma was gently mixed, and the Specimen Collection Loop was dipped into the tube of blood/plasma. The loop was completely filled with blood and then inserted into the vial. The loop was stirred into the Developer Solution and discarded. The flat pad of the device was immersed in the blood sample and results were recorded after 20 minutes.

### Experimental Results

Table 6 below shows the data collected using the kits for the detection of HCV. The bolded results indicate high or positive results.

TABLE 6

| DCN # | LOT# | HCV |
|---|---|---|
| 40 | BRH687840 | Negative |
| 41 | BRH687841 | Negative |
| 42 | BRH687842 | Negative |
| 43 | BRH687843 | Negative |
| 44 | BRH687844 | Negative |
| 45 | BRH687845 | Negative |
| 46 | BRH687846 | Negative |
| 47 | BRH687847 | Negative |
| 48 | BRH687848 | Negative |
| 49 | BRH687849 | Negative |
| 36 | BRH689636 | Positive |
| 37 | BRH689637 | Positive |

### Conclusions

The individuals numbered 36 and 37 tested positive for HCV.

### Example 3

### Rapid Assay for Cotinine

Introduction

The work was performed as part of the effort to demonstrate the rapid detection of cotinine in whole blood or saliva samples. The NicAlert Rapid Test kit was used to test 2 saliva samples presumed negative for cotinine.

### Materials and Methods

### Materials

The NicAlert Rapid test kit for cotinine is sold over the counter. The kit contains a cotinine test strip, a test card, a saliva collection funnel and saliva collection tube. The saliva samples used in the test are listed in the Table 7 below.

TABLE 7

| LOT# | Gender | AGE | RACE |
|---|---|---|---|
| Saliva | Male | 59 | Caucasian |
| Sample 1 | | | |
| Saliva | Female | 58 | Caucasian |
| Sample 2 | | | |

NicAlert Cotinine Test Kit from TestCountry.com, San Diego, Calif. Lot Number NM1077, Expiration November 2014 was used in the test.

### Methods

About 2 mls of saliva were collected from each of two presumably non-smoking donors into the saliva collection cup. The cap was added to the cup and 8 drops of saliva were squeezed from the cup onto the collection pad of the cotinine test strip. Results were read at 15 minute as per the kit instructions. Both strips were also read to assure that the tests ran correctly by checking that the green control area at the top of the strip had become clear.

### Experimental Results

Table 8 below shows the test results.

TABLE 8

| Sample # | Result |
|---|---|
| 1 | Negative |
| 2 | Negative |

Conclusion:

The two saliva samples tested negative for cotinine confirming the donors have not recently smoked tobacco.

The ordinarily skilled artisan can appreciate that the present invention can incorporate any number of the preferred features described above.

Further features and advantages of the present invention will become apparent to those of skill in the art in view of the detailed description of preferred embodiments which follows, when considered together with the attached drawings and claims.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A kit for assessing a life or health insurance applicant, which kit comprises at least two rapid test devices (e.g., singleplex or multiplex rapid test devices), said rapid test devices are configured to assess at least two analytes in a sample derived from a life or health insurance applicant, said at least two analytes being selected from the group consisting of a human immunodeficiency virus (HIV) antigen (e.g., a HIV polypeptide), a HIV polynucleotide, an anti-HIV antibody, a hepatitis C virus (HCV) antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), prostate-specific antigen (PSA), apolipoprotein A-1 (ApoA1), apolipoprotein B (ApoB), Hemoglobin A1c and high-sensitivity C-reactive protein (hsCRP), and preferably, said kit can be used for assessing a life or health insurance applicant at point of presence.

2. The kit of claim 1, which comprises at least 5 rapid test devices, and wherein the rapid test devices are configured to assess 5 analytes selected from the group consisting of an anti-HIV antibody, an anti-HCV antibody, cotinine or nicotine, cocaine or benzoylecgonine, and Hemoglobin A1c.

3. The kit of claim 1, wherein at least one of the rapid test devices is selected from the group consisting of a lateral flow test device, a flow through test device, a microfluidic test device, an immunochromatography test device, a single use cartridge or test device, a disposable test device, a self-contained test device, a point of care test device, a microsphere based test device, a nanoparticle based test device, a test strip device, a spot test device, a centrifugal device, and a pump based test device.

4. The kit of claim 1, wherein at least one of the rapid test devices can be used to conduct a test within about 12 hours.

5. The kit of claim 4, which comprises at least 5 lateral flow test devices, and wherein the lateral flow test devices are configured to assess 5 analytes selected from the group consisting of an anti-HIV antibody, an anti-HCV antibody, cotinine or nicotine, cocaine or benzoylecgonine, and Hemoglobin A1c.

6. A system for assessing a life or health insurance applicant, which system comprises the kit of claim 1.

7. The system of claim 6, which further comprises:
a) a means for assessing an additional health indicator of the life or health insurance applicant;
b) a means for obtaining at least two different types of samples from the life or health insurance applicant; and/or
c) machine-readable information, e.g., a bar code.

8. The system of claim 6, which further comprises:
a) a reader to assess the detectable signal;
b) a means for recording, storing and/or sending test results in an electronic format;
c) a software program and/or algorithm that collates and/or optimizes all data inputs such as assay results, BMI, application, release form, attending physician statement (APS), medical information bureau (MIB) record, motor vehicle record (MVR), credit report and prescription or transaction history record into one electronically transferrable or printable file; and/or
d) and/or an apparatus, software and/or algorism for forensic voice analysis that detects emotions in the human voice.

9. A lateral flow test device for assessing a life or health insurance applicant, which device is configured to assess at least two analytes in a sample derived from a life or health insurance applicant, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and
said device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to assess said at least two analytes in a sample, and the formation of said detectable signal requires the use of a detectable label.

10. The device of claim 9, which is configured to assess 5 analytes selected from the group consisting of an anti-HIV antibody, an anti-HCV antibody, nicotine or cocaine, cocaine or benzoylecgonine, and Hemoglobin A1c.

11. The device of claim 9, wherein a substance is dried on a portion of the matrix upstream from the test locations, the dried substance being capable of being moved by a liquid sample and/or a further liquid to the test locations and/or a control location to generate a detectable signal, the dried substance being at least one of the second binding reagent that binds to the analyte, the second binding reagent that binds to another binding reagent that binds to the analyte, the analyte or the analyte analog, each of the second binding reagent, analyte or analyte analog comprises a detectable label.

12. The device of claim 9, which is configured to receive at least two different types of samples, e.g., two different types of samples from the applicant.

13. The device of claim 12, wherein the test locations are in the same or different liquid flow pathway.

14. A system for assessing a life or health insurance applicant, which system comprises the device of claim 9.

15. The system of claim 14, which further comprises:
a) an instruction for using the system for assessing mortality and/or morbidity risk of the life insurance or health applicant, and/or making a decision on the insurance application from the life insurance applicant;
b) a means for assessing an additional health indicator of the life or health insurance applicant;
c) a means for assessing an identifier of the life or health insurance applicant;
d) a means for obtaining at least two different types of samples from the life or health insurance applicant;
e) a means for collecting more than one finger stick of blood from the life or health insurance applicant to increase the volume of blood sample to be able to run multiple assays on the blood sample; and/or
f) a finger stick device that simultaneously makes at least two punctures at once to increase the amount of blood sample volume needed to perform multiple assays on the blood sample; and/or
g) machine-readable information, e.g., a bar code.

16. The system of claim 14, which further comprises:
a) a reader to assess the detectable signal;
b) a means for recording, storing and/or sending test results in an electronic format;
c) a software program and/or algorithm that collates and/or optimizes all data inputs such as assay results, BMI, application, release form, attending physician statement (APS), medical information bureau (MIB) record, motor vehicle record (MVR), credit report and prescription or transaction history record into one electronically transferrable or printable file; and/or
d) and/or an apparatus, software and/or algorism for forensic voice analysis that detects emotions in the human voice.

17. A method for assessing a life or health insurance applicant, which method comprises:
a) assessing the presence, absence and/or amount of at least two analytes in a sample derived from a life or health insurance applicant using a rapid test, said at least two analytes being selected from the group consisting of a HIV antigen (e.g., a HIV polypeptide), a HIV polynucleotide, an anti-HIV antibody, a HCV antigen (e.g., a HCV polypeptide), a HCV polynucleotide, an anti-HCV antibody, a liver enzyme alanine transaminase (ALT), a liver enzyme aspartate transaminase (AST), nt-probnp (or NT-proBNP), probnp (or proBNP), cotinine, nicotine, cocaine, a metabolite of cocaine (e.g., benzoylecgonine), PSA, ApoA1, ApoB, Hemoglobin A1c and hsCRP, and
b) assessing an insurance application from said life or health insurance applicant based on the test results obtained in step a).

18. The method of claim 17, which is conducted using the kit of claim 1.

19. The method of claim 17, which is conducted using the system of claim 6.

20. The method of claim 17, which is conducted using the device of claim 9.

21. The method of claim 17, which is conducted using the system of claim 14.

22. The method of claim 17, wherein the liquid sample is a body fluid sample.

23. The method of claim 17, which comprises assessing 5 analytes selected from the group consisting of an anti-HIV antibody, an anti-HCV antibody, cotinine or nicotine, cocaine or benzoylecgonine and Hemoglobin A1c.

24. The method of claim 17, wherein the detectable signal is assessed by a reader.

25. The method of claim 17, wherein a sample derived from a life or health insurance applicant without fasting (e.g., food fasting) is used.

26. The method of claim 17, which further comprises:
a) assessing an additional health indicator of a life or health insurance applicant;
b) assessing an identifier of a life or health insurance applicant; and/or
c) conducting forensic voice analysis of the voice of an applicant.

27. The method of claim 17, which is conducted at a point of presence.

28. The method of claim 17, wherein the test results and collated information are transmitted to an insurance decision maker within about 24 hours from the time when a sample is obtained from the applicant.

29. The kit of claim 1, which further comprises a solid medium for collecting, drying and/or storing a sample derived from a life or health insurance applicant wherein said collected, dried and/or stored sample can be used in a test.

30. The system of claim 6, which further comprises a solid medium for collecting, drying and/or storing a sample derived from a life or health insurance applicant wherein said collected, dried and/or stored sample can be used in a test.

31. The device of claim 9, which further comprises a solid medium for collecting, drying and/or storing a sample derived from a life or health insurance applicant wherein said collected, dried and/or stored sample can be used in a test.

32. The system of claim 14 which further comprises a solid medium for collecting, drying and/or storing a sample derived from a life or health insurance applicant wherein said collected, dried and/or stored sample can be used in a test.

33. The method of claim 17, which further comprises collecting, drying and/or storing a sample derived from a life or health insurance applicant on or in a solid medium wherein said collected, dried and/or stored sample can be used in a test.

34. The system of claim 6, which further comprises a device or an instrument for conducting a thyroid test, a thyroid panel test, a test for a liver marker or disease, a metabolic panel test, a lipid test, a test for blood count, an autoimmune test, and/or an infectious disease test on a sample from a life or health insurance applicant.

35. The method of claim 17, which further comprises conducting a thyroid test, a thyroid panel test, a test for a liver marker or disease, a metabolic panel test, a lipid test, a test for blood count, an autoimmune test, and/or an infectious disease test on a sample from a life or health insurance applicant.
